(19) United States
(12) Patent Application Publication
(10) Pub. No.: US 2023/0248850 A1
(43) Pub. Date: Aug. 10, 2023

(54) GOLD MOLECULAR CLUSTERS AND METHODS OF USING SAME FOR NEAR-INFRARED IMAGING

(71) Applicant: The Board of Trustees of the Leland Stanford Junior University, Stanford, CA (US)

(72) Inventors: Hongjie Dai, Cupertino, CA (US); Ani Baghdasaryan, Redwood City, CA (US); Feifei Wang, Redwood City, CA (US)

(21) Appl. No.: 18/102,623

(22) Filed: Jan. 27, 2023

Related U.S. Application Data
(60) Provisional application No. 63/304,470, filed on Jan. 28, 2022.

(51) Int. Cl. A61K 49/00

(52) U.S. Cl. CPC A61K 49/0065

(57) ABSTRACT

Provided are gold molecular clusters functionalized with phosphorylcholine (PC) ligands. Also provided are compositions comprising the gold molecular clusters and methods of in vivo imaging of a tissue in a subject, the methods comprising administering a composition of the present disclosure to the subject, and performing NIR-I or NIR-II in vivo fluorescence imaging of the tissue. Also provided are kits comprising the gold molecular clusters and compositions of the present disclosure, as well as methods of synthesizing gold molecular clusters functionalized with PC ligands.

### CROSS-REFERENCE TO RELATED APPLICATIONS

This application claims the benefit of U.S. Provisional Patent Application No. 63/304,470, filed Jan. 28, 2022, which application is incorporated herein by reference in its entirety.

### STATEMENT OF GOVERNMENT SUPPORT

This invention was made with Government support under contract NS105737 awarded by the National Institutes of Health. The Government has certain rights in the invention.

### INTRODUCTION

Sentinel lymph nodes (SLN) are the primary tumor drainage nodes to which cancer metastasis first occur. The tumor cells disseminate from the peritumoral lymphatics to the SLN and then to distant nodes to initiate lymphatic spread of malignant tumor cells¹. SLN biopsy (SLNB) is a standard-of-care cancer staging modality and comprises the peritumoral administration of radioisotopes, dye tracers or a combination of the two for SLN identification². This is done by preoperatively administering common tracers of technetium-99 m isotope (for lymphoscintigraphy), a fluorescent NIR-I (700-900 nm) dye indocyanine green (ICG)^{3,4,5,6,7}, methylene blue (MB)^{8,9} or their combination and detecting the signals of the tracers drained to the SLNs. The introduction of lymphoscintigraphy in SLNB is thus far considered the “gold standard” in clinical oncology for assessing and staging breast, melanoma, head and neck cancer metastasis^{10,11,12}. High SLN detection rates were achieved in clinical trials with scintigraphy in conjunction with SPECT/CT and intraoperative administration of a secondary visual blue dye. The SLNs are usually visualized within 10-60 min (sometimes several hours), however, several risk factors do contribute to a mis-detection rate of 2-28%¹³. Disadvantages of lymphoscintigraphy include either scarcity of nuclear medicine facilities or lack of access to radiopharmaceuticals. Operations involving radio-activity pose certain risks to healthcare workers. Also, the radiological procedures are generally ruled out for some patient groups (e.g., pregnant women)¹⁴. Instead, as an alternative, cheaper and non-inferior tracer to lymphoscintigraphy, ICG has been widely pursued for LN imaging for breast, dermatological and oncological cancers¹⁵. Subsequent surgical excision and pathological examination of labeled lymph nodes affords an assessment of the presence and possible spread of cancer, providing a guidance to proper and efficient treatment¹⁶.

Since 2009¹⁷ in vivo one-photon fluorescence imaging of biological systems in the NIR-II window (1000-3000 nm) has led to non-invasive, real-time, and high-resolution imaging of biological structures (including lymph nodes) and processes at single cell and single vasculature level^{18,19,20,21,22}, complementing other imaging modalities including computed X-ray tomography (CT)²³, radio-imaging²⁴, photo-acoustic imaging²⁵ and magnetic resonance imaging (MRI)²⁶. NIR-II imaging guided surgical interventions/excisions are also actively pursued^{27,28,29}. Fluorescence imaging in the NIR-II window benefits from reduced light scattering by tissues³⁰ and suppressed tissue autofluorescence background signals³¹, affording higher sensitivity, higher temporal and spatial resolution at deeper penetration depths (sub-cm)^{18,19,20,21,22} than previous NIR-I imaging in the 800-900 nm wavelength range. A range of organic and inorganic NIR-II probes, such as donor-acceptor dyes^{32,33}, carbon nanotubes (CNTs)^{18,34}, quantum dots (QDs)^{20,35} and rear-earth down-conversion nanoparticles^{36,37} have been employed for NIR-II through-skin/-tissue imaging of blood vasculatures^{18,21,22,38} in studies of cardiovascular diseases and traumatic brain injury (TBI)^{19,39}, molecular imaging of cancers^{36,40} and assessing response to immunotherapy at the single-cell level in vivo^{19,22}. Lymph node imaging in the NIR-II window has also been pursued^{33,35,41}, but much work is still needed to further advance NIR-II probes to achieve high LN/background ratios, well-defined timing for probe administration/imaging, and high safety and rapid clearance.

Gold molecular clusters^{42,43,44,45} have attracted interest due to their molecular-like structures⁴⁶ and resulting properties⁴⁷, high stability⁴⁸ and importantly, safety and biocompatibility^{49,50,51}. Several gold clusters have shown photoluminescence extending beyond the UV-vis region of the spectrum to NIR^{52,53,54,55,56}. Water-soluble Au₂₅(GSH)₁₈ (GSH: glutathione) clusters emitting in the >1000 nm range were used for through-skull brain imaging and detection of cerebral blood vessels in lipopolysaccharides (LPS) induced brain injury and stroke in vivo⁵². Gold molecular clusters coated with glutathione ligands were also employed for NIR-II fluorescence imaging of bones taking advantage of efficient Au-GSH binding to the bone matrix⁵⁵. Anti-CD326 labeled⁵⁶ and folic acid capped PEGylated Au clusters loaded with chlorin e6 (Ce6) photosensitizer⁵⁴ showed excellent tumor penetration and retention in xenograft MCF-7 and MGC-803 tumor mouse models as well photodynamic therapy (PDT) effect with Ce6 loaded clusters⁵⁴. Despite this progress, thus far little has been done on lymph node imaging using NIR-II emitting Au molecular clusters.

### SUMMARY

Provided are gold molecular clusters functionalized with phosphorylcholine (PC) ligands. Also provided are compositions comprising the gold molecular clusters and methods of in vivo imaging of a tissue in a subject, the methods comprising administering a composition of the present disclosure to the subject, and performing NIR-I or NIR-II in vivo fluorescence imaging of the tissue. Also provided are kits comprising the gold molecular clusters and compositions of the present disclosure, as well as methods of synthesizing gold molecular clusters functionalized with PC ligands.

### BRIEF DESCRIPTION OF THE FIGURES

FIG. 1A-1G: A: Crystallographic representation of Au₂₅ cluster structure. Color codes of the elements: Au (0) in the core: yellow, Au (I) in the staple motif: orange, S in the staple motif: green. The structure was prepared using UCSF Chimera program (version 1.12) based on crystal structure data published in Reference 46. B: Postfunctionalization of Au-GSH cluster and schematic representation of Au-PC conjugate structure. For clarity, only one staple motif and adjacent gold core atoms are shown. For simplicity, the conjugation of PC ligand to glycine carboxylic group is omitted and it is only shown with γ-glutamate carboxylic functional group of GSH. C: UV-vis absorption and fluorescence spectra of Au-GSH cluster in aqueous phase. D: CryoEM micrograph of Au-GSH clusters with an average size of 1.64 nm±0.24 nm (n=3). E: Descriptive statistical analyses of particle size distribution of Au-GSH clusters obtained from cryoEM micrograph. F: ESI-MS spectrum of the Au-GSH cluster in negative ion mode from m/z 1000 to 3000: several negatively charged species of 5-8 were identified and the remaining peaks were small and attributed to impurity clusters/species. G: ESI-MS spectra of major peaks 5-8 with estimated sodium adducts were assigned to a common [Au₂₅(GS)₁₈+xNa-xH-zH]^{z} formula. a.u.: arbitrary units.

FIG. 2A-2F: Wide-field NIR-II fluorescence images (excited by an 808 nm laser at a power density of 70 mW/cm², exposure times 40 ms and 4 ms for Au and ICG, respectively, 1100 nm long pass filter) of intravenously (i.v.) injected A Au-PC conjugate (4×, ˜1.2 mg), B Au-GSH cluster (4×, ˜1.2 mg) and (C) ICG (50 μL, 50 mM) probes at different time points (six to seven weeks old female Balb/c, n=3). Biodistribution in major organs at 24 h post-injection of D Au-PC and E Au-GSH fluorescent probes. Error bars represent standard deviation (SD) of three repeated experiments. Bar graphs data presented as mean values±SD. F: Microanatomy of histological sections of hematoxylin and eosin (H&E)-stained major organs from healthy mouse and mouse injected with Au-PC conjugate 24 h postinjection (20× objective, scale bar is 100 μm).

FIG. 3A-3F: Wide-field NIR-II fluorescence images (excited by an 808 nm laser at a power density of 70 mW/cm², exposure times 20, 40 ms and 4 ms for Au-PC, Au-GSH and ICG, respectively, 1100 nm long pass filter) of (peri)intra-tumoral (i.t.) injected A Au-PC conjugate (1×, ˜300 μg), B Au-GSH cluster (1×, ˜300 μg) and C ICG (50 μL, 50 mM) probes into a mouse bearing 4T1 tumors on hindlimbs at different time points (six to seven weeks old female Balb/c, n=3). Normalized fluorescence intensities (left Y axes) and lymph node signal-to-background (LN/B) ratios (right Y axes) of inguinal lymph nodes (iLNs) up to six hours postinjection of D Au-PC conjugate, E Au-GSH cluster and F ICG fluorescent probes. Error bars represent the standard deviation (SD) of three repeated experiments. Data are presented as mean values±SD.

FIG. 4A-4D: A, C Rapid renal excretion profiles (a up to 24 h p.i.) and B, D biodistribution in major organs after 48 h of intra-tumoral (i.t.) administration of Au-PC and Au-GSH fluorescent probes into a mouse bearing 4T1 tumors on hindlimbs (n=3), respectively. The insets in a and c represent NIR-II fluorescence images (excited by an 808 nm laser at a power density of 70 mW/cm², exposure time 40 ms, 1100 nm long pass filter) of collected urine samples at different time points for Au-PC and Au-GSH, respectively. Error bars represent standard deviation (SD) of three repeated experiments. Data are presented as mean values±SD.

FIG. 5A-5F: Wide-field NIR-II fluorescence images (excited by an 808 nm laser at a power density of 70 mW/cm², exposure times 100 ms and 4 ms for Au and ICG, respectively, 1100 nm long pass filter) of bilateral subcutaneously (s.c.) injected A Au-PC conjugate (4×, ˜1.2 mg), B Au-GSH cluster (4∴, ˜1.2 mg) and C ICG (50 μL, 50 mM) probes at different time points (six to seven weeks old female Balb/c, n=3). Normalized fluorescence intensities (left Y axes) of Right (R) inguinal lymph nodes (iLNs) up to six hours postinjection and region of interest signal (ROI, right Y axes) around the injection site up to 24 h post-injection of Au-PC (D), Au-GSH (E) and ICG (F) fluorescent probes. Error bars represent standard deviation (SD) of three repeated experiments. Data are presented as mean values±SD.

FIG. 6A-6D: Wide-field fluorescence images (excited by an 808 nm laser at a power density of 70 mW/cm²) of intra-tumoral (i.t.) injected A 1× Au-PC conjugate and B ICG probes into a mouse bearing 4T1 tumor (six weeks old female Balb/c, n=3). The images present the right lateral view taken 30 min p.i and 3 h p.i for Au-PC and ICG respectively at different NIR-I and NIR-II windows. The exposure times for detecting >900 nm, >1100 nm, >1200 nm and >1300 nm emission were 25 ms, 20 ms, 90 ms and 400 ms for Au-PC and 0.4 ms, 3 ms, 15 ms, 100 ms for ICG respectively. C Fluorescence intensity cross-sectional profile of iLN after Au-PC administration. Locations of LN signal and background (B) on the line profiles are marked with arrows. (D) The comparison of lymph node signal-to-background (LN/B) ratios of Right (R) inguinal lymph node (iLNs) at different NIR-I and NIR-II sub-windows. Error bars represent standard deviation of three repeated experiments.

FIG. 7A-7B: Spectroscopic characterization of clusters: UV-vis spectra. (a) UV-vis spectra of Au-GSH cluster and Au-PC conjugate in water (0.6 μg/μL). The inset shows the spectra below 400 nm. (b) The comparison of OD at 250 nm for both clusters. Error bars represent standard deviation of three repeated experiments (different batches).

FIG. 8: Spectroscopic characterization of clusters: ATR-FTIR spectra. ATR-FTIR spectra of PC ligand (green trace), Au-GSH (black trace) and Au-PC (red trace). The aqueous solutions of PC ligand (200 μg, 20 μL), Au-GSH cluster (1×, 300 μg, 36 μL) and Au-PC (1×, 300 μg, 20 μL) were drop casted on diamond IRE and allowed to air dry. The IR spectra of a solid was measured in ATR mode. The spectra were recorded with a spectral resolution of 4 cm⁻¹, in the range 400-4000 cm⁻¹ and are scaled for better comparison.

FIG. 9A-9B: Microscopic characterization of Au-PC: cryoEM. (A) CryoEM micrograph of Au-PC conjugate with an average size of 1.65±0.22 nm (n=3). (B) Descriptive statistical analyses of particle size distribution of Au-PC conjugate obtained from cryoEM micrograph.

FIG. 10A-10C: Stability of Au-GSH and Au-PC clusters. (A) Photostability of the cluster upon continuous 808 nm laser irradiation at a power density of 35 mW/cm² for two hours. Almost complete recovery of the initial intensity after two hours of “laser-off” standing regime. PL stability of (B) Au-GSH cluster and (C) Au-PC conjugate before and after two weeks of incubation. Error bars represent standard deviation (SD) of four repeated experiments. Bar graphs data presented as mean values±SD. **: P≤0.01, ***: P≤0.001, Tukey's test (one-sided). a.u.: arbitrary units.

FIG. 11A-11B: Cell viability. Cell viability test of murine breast cancer 4T1 and colon cancer CT26 cells after 12 h of incubation with varying concentrations of Au-GSH (A) and Au-PC (B) probes. Experiments were conducted in triplicates. Error bars represent standard deviation of three repeated experiments.

FIG. 12A-12C: Serum protein binding test. (A) Schematic illustration of serum protein binding efficiency test with Au-GSH+FBS, Au-PC+FBS and ICG+FBS. The samples were incubated for 1 h at 37° C. followed by centrifugal filtration using Amicon 50 kDa centrifuge filters. (B) NIR-II images of Au-GSH+FBS and Au-PC+FBS filtrates and ICG+FBS retentate after filtration (excited by an 808 nm laser at a power density of 70 mW/cm², exposure time 10 ms, 1100 nm long pass filter). (C) The optical density (OD) of filtrates (in case of Au-GSH+FBS and Au-PC+FBS) and retentate (in case of ICG+FBS) at 808 nm compared to the OD before filtration. The corresponding binding efficiencies were calculated based on the OD of the initial solutions and after filtration.

FIG. 13A-13C: In vivo fluorescence imaging with intravenous injected Au-PC. Wide-field NIR-II fluorescence images (excited by an 808 nm laser at a power density of 70 mW/cm², exposure time 40 ms and 1100 nm long pass filter) of intravenously (i.v.) injected Au-PC conjugate probe into a mouse at different time points (six weeks old female Balb/c, n=3). (A) The images are presented through dorsal, right and left lateral views. (B) Rapid renal excretion profiles after i.v administration of Au-PC probe. The insets represent NIR-II fluorescence images (excited by an 808 nm laser at a power density of 70 mW/cm², exposure time 40 ms, 1100 nm long pass filter) of collected urine samples at different time points. Error bars represent standard deviation (SD) of three repeated experiments. Data are presented as mean values±SD. (C) The fluorescent signal in major organs after 24 h post-injection of Au-PC conjugate.

FIG. 14A-14C: In vivo fluorescence imaging with intravenous injected Au-GSH. Wide-field NIR-II fluorescence images (excited by an 808 nm laser at a power density of 70 mW/cm², exposure time 40 ms and 1100 nm long pass filter) of intravenously (i.v.) injected Au-GSH cluster probe into a mouse at different time points (six weeks old female Balb/c, n=3). (A) The images are presented through dorsal, right and left lateral views. (B) Rapid renal excretion profiles after i.v administration of Au-GSH probe. The insets represent NIR-II fluorescence images (excited by an 808 nm laser at a power density of 70 mW/cm², exposure time 40 ms, 1100 nm long pass filter) of collected urine samples at different time points. Error bars represent standard deviation (SD) of three repeated experiments. Data are presented as mean values±SD. (C) The fluorescent signal in major organs after 24 h post-injection of Au-GSH cluster.

FIG. 15A-15B: In vivo fluorescence imaging with intravenous injected ICG. Wide-field NIR-II fluorescence images (excited by an 808 nm laser at a power density of 70 mW/cm², exposure time 4 ms and 1100 nm long pass filter) of intravenously (i.v.) injected ICG dye into a mouse at different time points (seven weeks old female Balb/c, n=3, 50 μL from 50 μM stock solution). (A) The images are presented through dorsal, right and left lateral views. (B) The fluorescent signal in major organs after 24 h post-injection of ICG probe.

FIG. 16A-16B: In vivo fluorescence imaging with intra-tumoral injected 1× Au-PC. Wide-field NIR-II fluorescence images (excited by an 808 nm laser at a power density of 70 mW/cm², exposure time 20 ms and 1100 nm long pass filter) of peri-tumoral (i.t.) injected 1× Au-PC conjugate probe into a mouse bearing 4T1 tumor on the right hindlimb at different time points (six weeks old female Balb/c, n=3). (A) The images are presented through dorsal and ventral views. (B) The fluorescent signal in major organs after 24 h post-injection of Au-PC conjugate.

FIG. 17: In vivo fluorescence imaging with intra-tumoral injected Au-PC at different doses. Wide-field NIR-II fluorescence images (excited by an 808 nm laser at a power density of 70 mW/cm², exposure time 40 ms (A) and 20 ms (B) and 1100 nm long pass filter) of (A) intra-tumoral (i.t.) injected 4× dose of Au-PC (˜1.2 mg) into a mouse bearing 4T1 tumors on both hindlimbs at different time points (six weeks old female Balb/c, n=3). (B) Peri-tumoral injected ⅓× dose of Au-PC (˜100 ug) probe into a mouse bearing 4T1 tumor on the right hindlimb at different time points (six weeks old female Balb/c, n=3). The images are presented lateral views.

FIG. 18A-18F: In vivo fluorescence imaging with intra-tumoral injected Au-PC and Au-GSH: 4× dose. Wide-field NIR-II fluorescence images (excited by an 808 nm laser at a power density of 70 mW/cm², exposure time 40 ms and 1100 nm long pass filter) of intra-tumoral (i.t.) injected 4× doses (˜1.2 mg) of (a) Au-PC conjugate and (b) Au-GSH probe into a mouse bearing CT26 tumors, and (c) Au-GSH probe into a mouse bearing 4T1 tumors at different time points (six weeks old female Balb/c, n=3). The images are presented right (a and c) and left (b) lateral views. Normalized fluorescence intensities and lymph node signal-to-background (LN/B) ratios of inguinal lymph nodes (iLNs) post six hours injection of (d) Au-PC (CT26) and (e; CT26 and f; 4T1) Au-GSH fluorescent probes. Error bars represent standard deviation (SD) of three repeated experiments. Data are presented as mean values±SD.

FIG. 19A-19C: In vivo fluorescence imaging of aLN with intra-tumoral injected Au-PC. Wide-field NIR-II fluorescence images (excited by an 808 nm laser at a power density of 70 mW/cm², exposure times 20 ms (a) and 40 ms (b, c), 1100 nm long pass filter) of intra-tumoral (i.t.) injected (a) 1× dose and (b) 4× dose of Au-PC conjugate,

(c) Au-GSH clusters into a mouse bearing (a, c) 4T1 and (b) CT26 tumors at different time points (six weeks old female Balb/c, n=3). The images are presented ventral views. The location of axillary lymph nodes (aLNs) post 10 min and 30 min injection of probes is shown with arrows. Weak signal in aLN can be seen at 10 min p.i, which disappears after 30 min p.i.

FIG. 20A-20D: In vivo fluorescence imaging with intra-tumoral injected Au-GSH. Wide-field NIR-II fluorescence images (excited by an 808 nm laser at a power density of 70 mW/cm², exposure time 40 ms, 1100 nm long pass filter) of (peri)intra-tumoral (i.t.) injected Au-GSH cluster (1×, ˜300 μg) probe into a mouse bearing (A) 4T1 tumors and (C) CT26 tumors on hindlimbs at different time points (six to seven weeks old female Balb/c, n=3-4). (B, D) The fluorescent signal in major organs after 10 min post-injection of Au-GSH cluster.

FIG. 21A-21D: In vivo fluorescence imaging with intra-tumoral injected Au- PC. Wide-field NIR-II fluorescence images (excited by an 808 nm laser at a power density of 70 mW/cm², exposure time 40 ms, 1100 nm long pass filter) of (peri)intra-tumoral (i.t.) injected Au-PC conjugate (1×, ˜300 μg) probe into a mouse bearing (A) 4T1 tumors and (C) CT26 tumors on hindlimbs at different time points (six to seven weeks old female Balb/c, n=3). (B, D) The fluorescent signal in major organs after 30 min post-injection of Au-PC conjugate.

FIG. 22A-22F: Intra-tumoral injection of Au-PC and Au-GSH: biodistribution at draining lymph node peak point. Biodistribution of organs at highest lymph node draining time point (10 min p.i. for Au-GSH and 30 min p.i. for Au-PC). Normalized (by maximum detectable signal so the data here reflected the relative signal in various organs) fluorescent intensities of major organs imaged ex vivo (after sacrificing the mice and organ removal from the bodies) 10 min and 30 min post intratumoral injection of (a, b) Au-GSH (1×, ˜300 μg) and (c, d) Au-PC (1×, ˜300 μg) to mice bearing (A, C) 4T1 and (B, D) CT26 tumors on hindlimbs. Normalized fluorescent intensities of iLNs after (E) 10 min p.i. of 1× Au-GSH and (F) 30 min p.i. of 1× Au-PC probes. **: P≤0.01, Tukey's test (one-sided). Error bars represent standard deviation (SD) of three to four repeated experiments. Bar graphs data in (E) and (F) presented as mean values±SD.

FIG. 23A-23C: In vivo fluorescence imaging with subcutaneous injected Au-PC. Wide-field NIR-II fluorescence images (excited by an 808 nm laser at a power density of 70 mW/cm², exposure time 100 ms and 1100 nm long pass filter) of bilateral subcutaneously (s.c.) injected 4× dose of Au-PC conjugate probe at different time points (six weeks old female Balb/c, n=3). (A) The images are presented through dorsal, ventral and left lateral views. (B) Normalized fluorescence intensity and lymph node signal-to-background (LN/B) ratio of Left (L) inguinal lymph node (iLN) up to six hours post-injection of a fluorescent probe. Error bars represent standard deviation (SD) of three repeated experiments. Data are presented as mean values±SD. (C) The fluorescent signal in major organs after 24 h post-injection of Au-PC conjugate.

FIG. 24A-24C: In vivo fluorescence imaging with subcutaneous injected Au-GSH. Wide-field NIR-II fluorescence images (excited by an 808 nm laser at a power density of 70 mW/cm², exposure time 100 ms and 1100 nm long pass filter) of bilateral subcutaneously (s.c.) injected 4× dose of Au-GSH cluster probe at different time points (six weeks old female Balb/c, n=3). (A) The images are presented through dorsal, ventral and left lateral views. (B) Normalized fluorescence intensity and lymph node signal-to- background (LN/B) ratio of Left (L) inguinal lymph node (iLN) up to six hours post-injection of a fluorescent probe. Error bars represent standard deviation (SD) of three repeated experiments. Data are presented as mean values±SD. (C) The fluorescent signal in major organs 24 h post-injection of Au-GSH cluster.

FIG. 25A-25D: Excretion profiles and biodistribution after subcutaneous injection of Au-PC and Au-GSH. Rapid renal excretion profiles (A, C) and biodistribution in major organs (B, D) after 24 h of subcutaneous (s.c.) administration of Au-PC and Au-GSH fluorescent probes (n=3), respectively. Error bars represent standard deviation (SD) of three repeated experiments. Data are presented as mean values±SD.

FIG. 26A-26C: In vivo fluorescence imaging with subcutaneous injected ICG. Wide-field NIR-II fluorescence images (excited by an 808 nm laser at a power density of 70 mW/cm², exposure time 4 ms and 1100 nm long pass filter) of bilateral subcutaneously (s.c.) injected ICG probe at different time points (seven weeks old female Balb/c, n=3). (A) The images are presented through dorsal, ventral and left lateral views. (B) Normalized fluorescence intensity and lymph node signal-to-background (LN/B) ratio of Left (L) inguinal lymph node (iLN) up to six hours post-injection of a fluorescent probe. Error bars represent standard deviation (SD) of three repeated experiments. Data are presented as mean values±SD. (C) The fluorescent signal in major organs after 24 h post-injection of ICG.

FIG. 27A-27B: Long-term toxicity study: intravenous injected Au-GSH. (A) Schematic representation of experimental timeline. Au-GSH clusters were systematically administered to mice (starting from three weeks old female Balb/c, n=3) weekly followed by the blood collection on day 48. (B) Wide-field NIR-II fluorescence images (excited by an 808 nm laser at a power density of 70 mW/cm², exposure time 40 ms, 1100 nm long pass filter) of intravenously (i.v.) injected Au-GSH cluster (1×, ˜300 μg) probe at different time points.

FIG. 28A-28B: Long-term toxicity study: intravenous injected Au-PC. (A) Schematic representation of experimental timeline. Au-PC clusters were systematically administered to mice (starting from three weeks old female Balb/c, n=3) weekly followed by the blood collection on day 48. (B) Wide-field NIR-II fluorescence images (excited by an 808 nm laser at a power density of 70 mW/cm², exposure time 40 ms, 1100 nm long pass filter) of intravenously (i.v.) injected Au-PC cluster (1×, ˜300 μg) probe at different time points.

FIG. 29A-29B: Long-term toxicity study: subcutaneous injected Au-GSH. (A) Schematic representation of experimental timeline. Au-GSH clusters were systematically administered to mice (starting from three weeks old female Balb/c, n=3) weekly followed by the blood collection on day 47. (B) Wide-field NIR-II fluorescence images (excited by an 808 nm laser at a power density of 70 mW/cm², exposure time 40 ms, 1100 nm long pass filter) of subcutaneously (s.c.) injected Au-GSH cluster (1×, ˜300 μg) probe at different time points.

FIG. 30A-30B: Long-term toxicity study: subcutaneous injected Au-PC. (A) Schematic representation of experimental timeline. Au-PC clusters were systematically administered to mice (starting from three weeks old female Balb/c, n=3) weekly followed by the blood collection on day 47. (B) Wide-field NIR-II fluorescence images (excited by an 808 nm laser at a power density of 70 mW/cm², exposure time 40 ms, 1100 nm long pass filter) of subcutaneously (s.c.) injected Au-PC cluster (1×, ˜300 μg) probe at different time points.

FIG. 31A-31B: Long-term toxicity study: body weight gain vs time. The body weight of mice was measured every second day after systematic (A) intravenous (i.v.) and (B) subcutaneous (s.c.) administration of Au-GSH (1×, ˜300 μg) and Au-PC probes (1×, ˜300 μg) weekly followed by the blood collection on day 48 (i.v.) and 47 (s.c.). Mice treated with only saline were used as a control group (n=3 in each group). Error bars represent standard deviation (SD) of three repeated experiments. Data are presented as mean values±SD.

FIG. 32: Long-term toxicity study: CBC blood test. CBC blood test results of mice after intravenous (i.v.) and subcutaneous (s.c.) administration of Au-GSH (1×, ˜300 μg) and Au-PC probes (1×, ˜300 μg). The blood collection was done on day 48 (i.v.) and 47 (s.c.). Mice treated with only saline were used as a control group (n=3 in each group). The test results of white blood cells (WBC; K/μL), red blood cells (RBC; M/μL), hemoglobin (HGB; gm/dL), hematocrit (HCT; %), mean corpuscular volume (MCV; fL), mean corpuscular hemoglobin (MCH; pg), and mean corpuscular hemoglobin concentration (MCHC; g/dL), neutrophils (%) and lymphocytes (%) represent standard deviation (SD) of three repeated experiments. Bar graphs data presented as mean values±SD. The data were analyzed by Tukey's test (one-sided).

FIG. 33: Long-term toxicity study: histology. Micro-anatomy of histological sections of hematoxylin and eosin (H&E)-stained organs from mice after intravenous (i.v.) and subcutaneous (s.c.) administration of Au-GSH (1×, ˜300 μg) and Au-PC probes (1×, ˜300 μg). Mice treated with only saline were used as a control group (n=3 in each group). 20× objective, scale bar is 100 μm.

### DETAILED DESCRIPTION

Before the gold molecular clusters, compositions and methods of the present disclosure are described in greater detail, it is to be understood that the gold molecular clusters, compositions and methods are not limited to particular embodiments described, as such may, of course, vary. It is also to be understood that the terminology used herein is for the purpose of describing particular embodiments only, and is not intended to be limiting, since the scope of the gold molecular clusters, compositions and methods will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the gold molecular clusters, compositions and methods. The upper and lower limits of these smaller ranges may independently be included in the smaller ranges and are also encompassed within the gold molecular clusters, compositions and methods, subject to any specifically excluded limit in the stated range. Where the stated range includes one or both of the limits, ranges excluding either or both of those included limits are also included in the gold molecular clusters, compositions and methods.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the gold molecular clusters, compositions and methods belong. Although any gold molecular clusters, compositions and methods similar or equivalent to those described herein can also be used in the practice or testing of the gold molecular clusters, compositions and methods, representative illustrative gold molecular clusters, compositions and methods are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present gold molecular clusters, compositions and methods are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the gold molecular clusters, compositions and methods, which are, for clarity, described in the context of separate embodiments, may also be provided in combination in a single embodiment. Conversely, various features of the gold molecular clusters, compositions and methods, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present gold molecular clusters, compositions and methods and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### Gold Molecular Clusters Functionalized with Phosphorylcholine (PC) Ligands and Related Compositions

Aspects of the present disclosure include gold molecular clusters functionalized with phosphorylcholine (PC) ligands. As demonstrated in the Experimental section below, the gold molecular clusters exhibit ‘super-stealth’ behavior with little interactions with serum proteins, cells and tissues in vivo, which differs from the indocyanine green (ICG) dye. Subcutaneous injection of Au-PC allows lymph node mapping by NIR-II fluorescence imaging at a time of, e.g., ˜0.5-1 hour post-injection followed by rapid renal clearance. Preclinical NIR-II fluorescence LN imaging with Au-PC affords high signal to background ratios and high safety and biocompatibility. Details regarding the functionalized gold molecular clusters will now be provided.

In certain embodiments, the functionalized gold molecular clusters comprise on average from 8 to 300 gold atoms. For example, the functionalized gold molecular clusters may comprise on average 10 to 40, 15 to 35, or 20 to 30 (e.g., about 25) gold atoms.

In some instances, the gold molecular clusters are functionalized with the PC ligands via covalent linkage between the PC ligands and the gold molecular clusters. According to some embodiments, the PC ligands are covalently linked to thiol molecules on the gold molecular clusters. In certain embodiments, the thiol molecules comprise glutathione (GSH). In some instances, the thiol molecules comprise cysteines. A non-limiting example approach for covalently linking the PC ligands and gold molecular clusters is described in detail in the Experimental section below.

According to some embodiments, the gold molecular clusters functionalized with PC ligands are biocompatible. As used herein, “biocompatible” means the ability of a material to perform the intended function of an embodiment of the present disclosure without eliciting undesirable local or systemic effects on the recipient. In certain embodiments, the gold molecular clusters functionalized with PC ligands are non-toxic upon administration to a subject.

Aspects of the present disclosure further include compositions. In some instances, provided are compositions comprising the gold molecular clusters functionalized with PC ligands of the present disclosure, e.g., any of the gold molecular clusters functionalized with PC ligands described elsewhere herein.

In certain aspects, the compositions include gold molecular clusters functionalized with PC ligands of the present disclosure present in a liquid medium. The liquid medium may be an aqueous liquid medium, such as water, a buffered solution, or the like. One or more additives such as a salt (e.g., NaCl, MgCl₂, KCl, MgSO₄), a buffering agent (a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.), a solubilizing agent, a detergent (e.g., a non-ionic detergent such as Tween-20 etc.), a ribonuclease inhibitor, glycerol, a chelating agent, and the like may be present in such compositions.

The compositions of the present disclosure may be formulated for administration to a subject. For example, the composition may be formulated for parenteral administration to the subject. Examples of parenteral administration include intravenous, intra-arterial, subcutaneous, intra-muscular, intra-dermal, intra-peritoneal, intra-vitreal, intra-tumoral, and peri-tumoral administration.

The compositions may include an effective amount of the gold molecular clusters functionalized with PC ligands. As used herein, an “effective amount” is meant a dosage sufficient to produce a desired result, e.g., a dosage sufficient to perform NIR-I (800-1000 nm) or NIR-II (1000-1700 nm) in vivo fluorescence imaging of a tissue according to the methods of the present disclosure. An effective amount can be administered in one or more administrations.

The gold molecular clusters functionalized with PC ligands of the present disclosure can be incorporated into a variety of formulations for administration to a subject. More particularly, the gold molecular clusters functionalized with PC ligands can be formulated into compositions by combination with appropriate, pharmaceutically acceptable excipients or diluents, and may be formulated into preparations in solid, semi-solid, liquid or gaseous forms, such as tablets, capsules, powders, granules, ointments, solutions, injections, inhalants and aerosols.

Formulations of the gold molecular clusters functionalized with PC ligands of the present disclosure suitable for administration to a subject (e.g., suitable for human administration) are generally sterile and may further be free of detectable pyrogens or other contaminants contraindicated for administration to a patient according to a selected route of administration.

In pharmaceutical dosage forms, the gold molecular clusters functionalized with PC ligands may also be used alone or in appropriate association, as well as in combination, with other pharmaceutically active compounds, e.g., an anti-cancer agent (including but not limited to small molecule anti-cancer agents), an immune checkpoint inhibitor, and any combination thereof. The following methods and carriers/excipients are merely examples and are in no way limiting.

For oral preparations, the gold molecular clusters functionalized with PC ligands can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The gold molecular clusters functionalized with PC ligands can be formulated for parenteral (e.g., intravenous, intra-arterial, subcutaneous, intra-muscular, intra-dermal, intra-peritoneal, intra-vitreal, intra-tumoral, and peri-tumoral, etc.) administration. In some instances, the gold molecular clusters functionalized with PC ligands are formulated for injection by dissolving, suspending or emulsifying the conjugate in an aqueous or non-aqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Compositions that include the gold molecular clusters functionalized with PC ligands may be prepared by mixing the gold molecular clusters functionalized with PC ligands having the desired degree of purity with optional physiologically acceptable carriers, excipients, stabilizers, surfactants, buffers and/or tonicity agents. Acceptable carriers, excipients and/or stabilizers are nontoxic to recipients at the dosages and concentrations employed, and include buffers such as phosphate, citrate, and other organic acids; antioxidants including ascorbic acid, glutathione, cysteine, methionine and citric acid; preservatives (such as ethanol, benzyl alcohol, phenol, m-cresol, p-chlor-m-cresol, methyl or propyl parabens, benzalkonium chloride, or combinations thereof); amino acids such as arginine, glycine, ornithine, lysine, histidine, glutamic acid, aspartic acid, isoleucine, leucine, alanine, phenylalanine, tyrosine, tryptophan, methionine, serine, proline and combinations thereof; monosaccharides, disaccharides and other carbohydrates; low molecular weight (less than about 10 residues) polypeptides; proteins, such as gelatin or serum albumin; chelating agents such as EDTA; sugars such as trehalose, sucrose, lactose, glucose, mannose, maltose, galactose, fructose, sorbose, raffinose, glucosamine, N-methylglucosamine, galactosamine, and neuraminic acid; and/or non-ionic surfactants such as Tween, Brij Pluronics, Triton-X, or polyethylene glycol (PEG).

The compositions may be in a liquid form, a lyophilized form or a liquid form reconstituted from a lyophilized form, wherein the lyophilized preparation is to be reconstituted with a sterile solution prior to administration. The standard procedure for reconstituting a lyophilized composition is to add back a volume of pure water (typically equivalent to the volume removed during lyophilization); however solutions comprising antibacterial agents may be used for the production of pharmaceutical compositions for parenteral administration.

An aqueous formulation of the gold molecular clusters functionalized with PC ligands may be prepared in a pH-buffered solution, e.g., at pH ranging from about 4.0 to about 7.0, or from about 5.0 to about 6.0, or alternatively about 5.5. Examples of buffers that are suitable for a pH within this range include phosphate-, histidine-, citrate-, succinate-, acetate-buffers and other organic acid buffers. The buffer concentration can be from about 1 mM to about 100 mM, or from about 5 mM to about 50 mM, depending, e.g., on the buffer and the desired tonicity of the formulation.

A tonicity agent may be included in the formulation to modulate the tonicity of the formulation. Example tonicity agents include sodium chloride, potassium chloride, glycerin and any component from the group of amino acids, sugars as well as combinations thereof. In some embodiments, the aqueous formulation is isotonic, although hypertonic or hypotonic solutions may be suitable. The term “isotonic” denotes a solution having the same tonicity as some other solution with which it is compared, such as physiological salt solution or serum. Tonicity agents may be used in an amount of about 5 mM to about 350 mM, e.g., in an amount of 100 mM to 350 mM.

A surfactant may also be added to the formulation to reduce aggregation and/or minimize the formation of particulates in the formulation and/or reduce adsorption. Example surfactants include polyoxyethylensorbitan fatty acid esters (Tween), polyoxyethylene alkyl ethers (Brij), alkylphenylpolyoxyethylene ethers (Triton-X), polyoxyethylene-polyoxypropylene copolymer (Poloxamer, Pluronic), and sodium dodecyl sulfate (SDS). Examples of suitable polyoxyethylenesorbitan-fatty acid esters are polysorbate 20, (sold under the trademark Tween 20™) and polysorbate 80 (sold under the trademark Tween 80™). Examples of suitable polyethylene-polypropylene copolymers are those sold under the names Pluronic® F68 or Poloxamer 188™. Examples of suitable Polyoxyethylene alkyl ethers are those sold under the trademark Brij™. Example concentrations of surfactant may range from about 0.001% to about 1% w/v.

A lyoprotectant may also be added in order to protect the gold molecular clusters functionalized with PC ligands against destabilizing conditions during a lyophilization process. For example, known lyoprotectants include sugars (including glucose and sucrose); polyols (including mannitol, sorbitol and glycerol); and amino acids (including alanine, glycine and glutamic acid). Lyoprotectants can be included in an amount of about 10 mM to 500 nM.

In some embodiments, the composition includes the gold molecular clusters functionalized with PC ligands of the present disclosure, and one or more of the above-identified agents (e.g., a surfactant, a buffer, a stabilizer, a tonicity agent) and is essentially free of one or more preservatives, such as ethanol, benzyl alcohol, phenol, m-cresol, p-chlor-m-cresol, methyl or propyl parabens, benzalkonium chloride, and combinations thereof. In other embodiments, a preservative is included in the formulation, e.g., at concentrations ranging from about 0.001 to about 2% (w/v).

### In Vivo Imaging Methods

Aspects of the present disclosure also include methods of in vivo imaging of a tissue in a subject. In certain embodiments, the methods comprise administering any of the compositions of the present disclosure to the subject, and performing NIR-I (800-1000 nm) or NIR-II (1000-1700 nm) in vivo fluorescence imaging of the tissue.

According to some embodiments, the administering is by parenteral administration to the subject. Non-limiting examples of parenteral routes of administration that find use when practicing the methods of the present disclosure include intravenous, subcutaneous, intra-muscular, intra-dermal, intraperitoneal, intravitreal administration, intra-tumoral, and peri-tumoral administration.

In some instances, performing NIR-I or NIR-II in vivo fluorescence imaging of the tissue comprises detecting <1000 nm or >1000 nm fluorescence under 660 nm, 740 nm or 808 nm laser or light-emitting diode (LED) excitation. For example, in certain embodiments, performing NIR-II in vivo fluorescence imaging of the tissue comprises imaging the tissue in the >1000 nm, >1100 nm, >1200 nm or >1300 nm NIR-II window. According to some embodiments, performing NIR-I or NIR-II in vivo fluorescence imaging of the tissue comprises exciting the gold molecular clusters at a wavelength of from 600 nm to 850 nm, optionally at a wavelength of about 660 nm or about 808 nm. Non-limiting examples of suitable excitation devices include a diode laser, an LED, or the like.

In certain embodiments, the excitation is performed at a power density of from 2 to 100 mW/cm². For example, the excitation may be performed at a power density of from 60 to 80 mW/cm², optionally at a power density of about 70 mW/cm².

Non-limiting examples of approaches, devices and settings for performing NIR-I (800-1000 nm) or NIR-II (1000-1700 nm) in vivo fluorescence imaging of a tissue in a subject according to the methods of the present disclosure are described in detail in the Experimental section herein.

The methods may be performed for in vivo fluorescence imaging of any tissue of interest. Non-limiting examples of tissues that may be imaged according to the methods of the present disclosure include skin, brain, heart, kidney, liver, stomach, large intestine, lungs, and/or the like. According to some embodiments, the tissue is from an organ system selected from adrenal glands, anus, appendix, bladder (urinary), bone, bone marrow, brain, bronchi, diaphragm, ears, esophagus, eye, fallopian tube, gallbladder, genitals, heart, hypothalamus, joints, kidney, large intestine, larynx, liver, lung, lymph node, mammary gland, mesentery, mouth, nasal cavity, nose, ovaries, pancreas, pineal gland, parathyroid gland, pharynx, pituitary gland, prostate, rectum, salivary gland, skeletal muscle, smooth muscle, skin, small intestine, spinal cord, spleen, stomach, teeth, thymus gland, thyroid, trachea, tongue, ureter, urethra, ligament, tendon, hair, vestibular system, placenta, testes, vas deferens, seminal vesicles, bulbourethral glands, parathyroid gland, thoracic duct, arteries, veins, capillaries, lymphatic vessels, tonsils, neurons, subcutaneous tissue, olfactory epithelium (nose), cerebellum, and any combination thereof.

In some embodiments, the tissue is a lymph node. For example, as will be appreciated upon review of the present disclosure including the Experimental section herein, the subject may have cancer, and the lymph node may be a sentinel lymph node (SLN). Sentinel lymph nodes (SLN) are the primary tumor drainage nodes to which cancer metastasis first occur. The tumor cells disseminate from the peritumoral lymphatics to the SLN and then to distant nodes to initiate lymphatic spread of malignant tumor cells. SLN biopsy (SLNB) is a standard-of-care cancer staging modality.

When the tissue is an SLN, the methods may further comprise, subsequent to performing the NIR-I or NIR-II in vivo fluorescence imaging of the SLN, performing a biopsy on the SLN to assess for cancer metastasis. In some instances, such methods further comprise resecting (cutting out) the SLN when the assessment indicates the presence of cancer metastasis.

According to some embodiments, the tissue is a tumor. “Tumor”, as used herein, refers to all neoplastic cell growth and proliferation, whether malignant or benign, and all pre-cancerous and cancerous cells and tissues. The terms “cancer” and “cancerous” refer to or describe the physiological condition in mammals that is typically characterized by unregulated cell growth/proliferation. Examples of cancers that may be imaged (and optionally, resected) using the subject methods include, but are not limited to, carcinoma, lymphoma, blastoma, and sarcoma. In certain embodiments, when the cancer is a carcinoma, the carcinoma is a basal cell carcinoma, squamous cell carcinoma, renal cell carcinoma, ductal carcinoma in situ (DCIS), invasive ductal carcinoma, or adenocarcinoma.

Accordingly, in some embodiments, the subject comprises a cancerous tissue (e.g., a tumor) which is desired to be imaged using the methods of the present disclosure (and optionally, resected from the subject), and the cancerous tissue is a tissue from renal cancer; kidney cancer; glioblastoma multiforme; metastatic breast cancer; breast carcinoma; breast sarcoma; neurofibroma; neurofibromatosis; pediatric tumors; neuroblastoma; malignant melanoma; carcinomas of the epidermis; leukemias such as but not limited to, acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone cancer and connective tissue sarcomas such as but not limited to bone sarcoma, myeloma bone disease, multiple myeloma, cholesteatoma-induced bone osteosarcoma, Paget's disease of bone, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangio sarcoma, neurilemmoma, rhabdomyosarcoma, and synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, and primary brain lymphoma; breast cancer including but not limited to adenocarcinoma, lobular (small cell) carcinoma, intraductal carcinoma, medullary breast cancer, mucinous breast cancer, tubular breast cancer, papillary breast cancer, Paget's disease (including juvenile Paget's disease) and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and ciliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; cervical carcinoma; esophageal cancers such as but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; colorectal cancer, KRAS mutated colorectal cancer; colon carcinoma; rectal cancers; liver cancers such as but not limited to hepatocellular carcinoma and hepatoblastoma, gallbladder cancers such as adenocarcinoma; cholangiocarcinomas such as but not limited to papillary, nodular, and diffuse; lung cancers such as KRAS-mutated non-small cell lung cancer, non-small cell lung cancer, squamous cell carcinoma (epidermoid carcinoma), adenocarcinoma, large-cell carcinoma and small-cell lung cancer; lung carcinoma; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, androgen-independent prostate cancer, androgendependent prostate cancer, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acrallentiginous melanoma; kidney cancers such as but not limited to renal cell cancer, adenocarcinoma, hypernephroma, fibrosarcoma, transitional cell cancer (renal pelvis and/or uterer); renal carcinoma; Wilms' tumor; and bladder cancers such as but not limited to transitional cell carcinoma, squamous cell cancer, adenocarcinoma, carcinosarcoma. In some embodiments, the cancer is myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma, or papillary adenocarcinomas.

According to some embodiments, when the tissue is a tumor, the method comprises administering the composition via intra-tumor and/or peri-tumor injection, allowing the gold molecular clusters functionalized with PC ligands to infiltrate the tumor, and performing the NIR-I or NIR-II in vivo fluorescence imaging of the tumor. Such methods optionally further comprise resecting the tumor guided by the NIR-I or NIR-II in vivo fluorescence imaging of the tumor.

In certain embodiments, the NIR-I or NIR-II in vivo fluorescence imaging of the tissue is performed within 3 hours of administration of the composition, optionally within 2 hours of administration of the composition. For example, the NIR-I or NIR-II in vivo fluorescence imaging of the tissue may be performed within 2 hours of administration of the composition, optionally within 1 hour of administration of the composition. In some instances, the NIR-I or NIR-II in vivo fluorescence imaging of the tissue is performed within 30 minutes of administration of the composition, optionally within 20 minutes, with 10 minutes, or within 5 minutes of administration of the composition.

According to some embodiments, the gold molecular clusters functionalized with PC ligands are renally excreted from the subject within 3 days of administration of the composition, optionally within 2 days or within 1 day of administration of the composition.

In some instances, the gold molecular clusters functionalized with PC ligands are biocompatible. According to some embodiments, the gold molecular clusters functionalized with PC ligands are non-toxic to the subject.

### Kits

Aspects of the present disclosure further include kits. The kits of the present disclosure may include any of the reagents, gold molecular clusters functionalized with PC ligands, imaging devices, and/or the like, that find use in making the gold molecular clusters functionalized with PC ligands, and/or using compositions comprising the gold molecular clusters functionalized with PC ligands to perform any of the methods of the present disclosure.

In certain embodiments, provided are kits that comprise any of the compositions of the present disclosure, and instructions for administering the composition to a subject for in vivo imaging of a tissue in the subject.

The kits of the present disclosure may include a quantity of the compositions, present in unit dosages, e.g., ampoules, or a multi-dosage format. As such, in certain embodiments, the kits may include one or more (e.g., two or more) unit dosages (e.g., ampoules) of a composition that includes the gold molecular clusters functionalized with PC ligands of the present disclosure. The term “unit dosage”, as used herein, refers to physically discrete units suitable as unitary dosages for human and animal subjects, each unit containing a predetermined quantity of the composition calculated in an amount sufficient to produce the desired effect. The amount of the unit dosage depends on various factors, such as the particular gold molecular clusters functionalized with PC ligands employed, the effect to be achieved, and the pharmacodynamics associated with the the gold molecular clusters functionalized with PC ligands, in the individual. In yet other embodiments, the kits may include a single multi dosage amount of the composition.

The instructions (e.g., instructions for use (IFU)) included in the kits may be recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., portable flash drive, DVD, CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, the means for obtaining the instructions is recorded on a suitable substrate.

### Methods of Synthesizing Gold Molecular Clusters Functionalized with PC Ligands

Aspects of the present disclosure further include methods of synthesizing gold molecular clusters functionalized with PC ligands. In certain embodiments, such methods comprise functionalizing gold molecular clusters with PC ligands.

According to some embodiments, functionalizing the gold molecular clusters with PC ligands comprises covalently linking the PC ligands to the gold molecular clusters. In some instances, the functionalizing comprises covalently linking the PC ligands to thiol molecules on the gold molecular clusters. For example, the thiol molecules may comprise GSH. In certain embodiments, the gold molecular clusters are gold-glutathione (Au-GSH) clusters, and the functionalizing comprises covalently linking the PC ligands to the Au-GSH clusters by 1-ethyl-3-(3-dimethylaminopropyl)carbodiimide (EDC)/N-hydroxysulfosuccinimide (NHS) chemistry. In certain embodiments, the thiol molecules comprise cysteines. In some instances, the functionalizing comprises covalently linking thiolated PC to the gold molecular clusters.

Non-limiting examples of approaches for synthesizing gold molecular clusters functionalized with PC ligands according to the synthetic methods of the present disclosure are described in detail in the Experimental section below.

Notwithstanding the appended claims, the present disclosure is also defined by the following embodiments:

1. Gold molecular clusters functionalized with phosphorylcholine (PC) ligands.
2. The gold molecular clusters of embodiment 1, wherein the gold molecular clusters comprise on average from 8 to 300 gold atoms.
3. The gold molecular clusters of embodiment 2, wherein the gold molecular clusters comprise on average from 20 to 30 gold atoms.
4. The gold molecular clusters of embodiment 3, wherein the gold molecular clusters comprise on average about 25 gold atoms.
5. The gold molecular clusters of any one of embodiments 1 to 4, wherein the gold molecular clusters are functionalized with the PC ligands via covalent linkage between the PC ligands and the gold molecular clusters.
6. The gold molecular clusters of embodiment 5, wherein the PC ligands are covalently linked to thiol molecules on the gold molecular clusters.
7. The gold molecular clusters of embodiment 6, wherein the thiol molecules comprise glutathione (GSH).
8. The gold molecular clusters of embodiment 6, wherein the thiol molecules comprise cysteines.
9. The gold molecular clusters of any one of embodiments 1 to 8, wherein the gold molecular clusters functionalized with PC ligands are biocompatible.
10. The gold molecular clusters of any one of embodiments 1 to 9, wherein the gold molecular clusters functionalized with PC ligands are non-toxic upon administration to a subject.
11. A composition comprising the gold molecular clusters of any one of embodiments 1 to 10.
12. The composition of embodiment 11, wherein the composition is formulated for administration to a subject.
13. The composition of embodiment 12, wherein the composition is formulated for parenteral administration to a subject.
14. The composition of embodiment 13, wherein the composition is formulated for intravenous, subcutaneous, intra-muscular, intra-dermal, intraperitoneal or intravitreal administration to a subject.
15. The composition of embodiment 13, wherein the composition is formulated for intra-tumoral and/or peri-tumoral administration to a subject having cancer.
16. A method of in vivo imaging of a tissue in a subject, the method comprising:

administering the composition of any one of embodiments 11 to 15 to the subject; and

performing NIR-I (800-1000 nm) or NIR-II (1000-1700 nm) in vivo fluorescence imaging of the tissue.

17. The method according to embodiment 16, wherein the administering is by parenteral administration to the subject.
18. The method according to embodiment 17, wherein the administering is by intravenous, subcutaneous, intra-muscular, intra-dermal, intraperitoneal or intravitreal administration to the subject.
19. The method according to embodiment 17, wherein the subject has cancer, and wherein the administering is by intra-tumoral and/or peri-tumoral administration to the subject.
20. The method according to any one of embodiments 16 to 19, wherein performing NIR-I or NIR-II in vivo fluorescence imaging of the tissue comprises detecting <1000 nm or >1000 nm fluorescence under 660 nm, 740 nm or 808 nm laser or LED excitation.
21. The method according to any one of embodiments 16 to 20, wherein performing NIR-II in vivo fluorescence imaging of the tissue comprises imaging the tissue in the >1000 nm, >1100 nm, >1200 nm or >1300 nm NIR-II window.
22. The method according to embodiment 21, wherein performing NIR-I or NIR-II in vivo fluorescence imaging of the tissue comprises exciting the gold molecular clusters at a wavelength of from 600 nm to 850 nm, optionally at a wavelength of about 660 nm or about 808 nm.
23. The method according to embodiment 22, wherein the excitation is performed using a diode laser or LED.
24. The method according to embodiment 22 or embodiment 23, wherein the excitation is performed at a power density of from 2 to 100 mW/cm².
25. The method according to embodiment 24, wherein the excitation is performed at a power density of from 60 to 80 mW/cm², optionally at a power density of about 70 mW/cm².
26. The method according to any one of embodiments 16 to 25, wherein the tissue is a lymph node.
27. The method according to embodiment 26, wherein the subject has cancer, and wherein the lymph node is a sentinel lymph node (SLN).
28. The method according to embodiment 27, further comprising, subsequent to performing NIR-I or NIR-II in vivo fluorescence imaging of the SLN, performing a biopsy on the SLN to assess for cancer metastasis.
29. The method according to embodiment 28, further comprising resecting the SLN when the assessment indicates the presence of cancer metastasis.
30. The method according to any one of embodiments 16 to 25, wherein the tissue is a tumor.
31. The method according to embodiment 30, wherein the method comprises administering the composition via intra-tumor and/or peri-tumor injection, allowing the gold molecular clusters functionalized with PC ligands to infiltrate the tumor, and performing the NIR-I or NIR-II in vivo fluorescence imaging of the tumor.
32. The method according to embodiment 31, further comprising resecting the tumor guided by the NIR-I or NIR-II in vivo fluorescence imaging of the tumor.
33. The method according to any one of embodiments 16 to 32, wherein the NIR-I or NIR-II in vivo fluorescence imaging of the tissue is performed within 3 hours of administration of the composition, optionally within 2 hours of administration of the composition.
34. The method according to any one of embodiments 16 to 32, wherein the NIR-I or NIR-II in vivo fluorescence imaging of the tissue is performed within 2 hours of administration of the composition, optionally within 1 hour of administration of the composition.
35. The method according to any one of embodiments 16 to 32, wherein the NIR-I or NIR-II in vivo fluorescence imaging of the tissue is performed within 30 minutes of administration of the composition, optionally within 20 minutes, with 10 minutes, or within 5 minutes of administration of the composition.
36. The method according to any one of embodiments 16 to 35, wherein the gold molecular clusters functionalized with PC ligands are renally excreted from the subject within 3 days of administration of the composition, optionally within 2 days or within 1 day of administration of the composition.
37. The method according to any one of embodiments 16 to 36, wherein the gold molecular clusters functionalized with PC ligands are biocompatible.
38. The method according to any one of embodiments 16 to 37, wherein the gold molecular clusters functionalized with PC ligands are non-toxic to the subject.
39. A kit comprising:

the composition of any one of embodiments 12 to 15; and

instructions for administering the composition to a subject for in vivo imaging of a tissue in the subject.

40. The kit of embodiment 39, wherein the composition is present in two or more unit dosages.
41. A method of synthesizing gold molecular clusters functionalized with PC ligands, the method comprising functionalizing gold molecular clusters with PC ligands.
42. The method according to embodiment 41, wherein the functionalizing comprises covalently linking the PC ligands to the gold molecular clusters.
43. The method according to embodiment 42, wherein the functionalizing comprises covalently linking the PC ligands to thiol molecules on the gold molecular clusters.
44. The method according to embodiment 43, wherein the thiol molecules comprise GSH.
45. The method according to embodiment 44, wherein the gold molecular clusters are gold-glutathione (Au-GSH) clusters, and wherein the functionalizing comprises covalently linking the PC ligands to the Au-GSH clusters by 1-ethyl-3-(3-dimethylaminopropyl)carbodiimide (EDC)/N-hydroxysulfosuccinimide (NHS) chemistry.
46. The method according to embodiment 43, wherein the thiol molecules comprise cysteines.
47. The method according to embodiment 42, wherein the functionalizing comprises covalently linking thiolated PC to the gold molecular clusters.

The following examples are offered by way of illustration and not by way of limitation.

### EXPERIMENTAL

### Introduction

Sentinel lymph node (SLN) imaging and biopsy (SLNB) is important to clinical assessment of cancer metastasis, performed by using radioisotopes (lymphoscintigraphy), visual dye (e.g., methylene blue), fluorescent tracers (ICG) or a combination of these probes. The search for novel lymphographic tracers that provide higher accuracy and efficacy for the detection of SLN with faster clearance kinetics is still an ongoing challenge. Described herein is the development of gold molecular clusters (Au₂₅ on average) functionalized by phosphorylcholine (PC) ligands (Au-PC) for NIR-II (1000-3000 nm) fluorescence imaging of draining lymph nodes in 4T1 murine breast cancer and CT26 colon cancer tumor mouse models. The Au-phosphorylcholine (Au-PC) probes exhibited ‘super-stealth’ behavior with little interactions with serum proteins, cells and tissues in vivo, which differed from the isocyanine green (ICG) dye and allowed lymph node mapping within a window of minutes to 1-2 hours post injection followed by rapid renal clearance. In vivo fluorescence imaging of Au-PC labeled LNs in the >1000 nm range benefited from reduced light scattering/autofluorescence and a much lower degree of probe diffusing into surrounding tissues than ICG, affording high LN signal to background ratios. Since gold is widely accepted as a safe element, phosphorylcholine is the polar group on phospholipids on cells membranes and highly biocompatible, Au-PC provides a new type of high performance clinical lymph node imaging in the NIR-II window.

Described herein is the synthesis of Au-GSH molecular clusters in an aqueous solution followed by modification of Au-GSH by covalent conjugation of GSH to 4-aminophenylphospohryl choline (p-APPC or PC in short) ligands. Phosphorylcholine and derivatives are highly biocompatible in vitro and in vivo⁴⁹⁻⁵¹, well known to impart high resistance to nonspecific protein interactions on solid surfaces such as graphene oxide thin film⁵² and planar gold surfaces⁵³. The resulting Au-PC clusters were found to behave as ‘super-stealth’ probes in vivo without binding to serum proteins like ICG or taken up by cells like the parent Au-GSH clusters by dendritic cells,⁵⁴ allowing imaging of mouse lymph nodes within minutes of intra-tumoral or subcutaneous injection. The Au-PC clusters showed little retention at the injection site, which differed from many nanomaterials, and reached near 100% renal excretion from the body within 24 h. The results indicate the utility of Au-PC molecular clusters for improved NIR-II fluorescence imaging for human use, e.g., improved NIR-II fluorescence lymph node imaging for human use in the clinic.

### Example 1—Gold Nanocluster Synthesis, Functionalization and Characterization

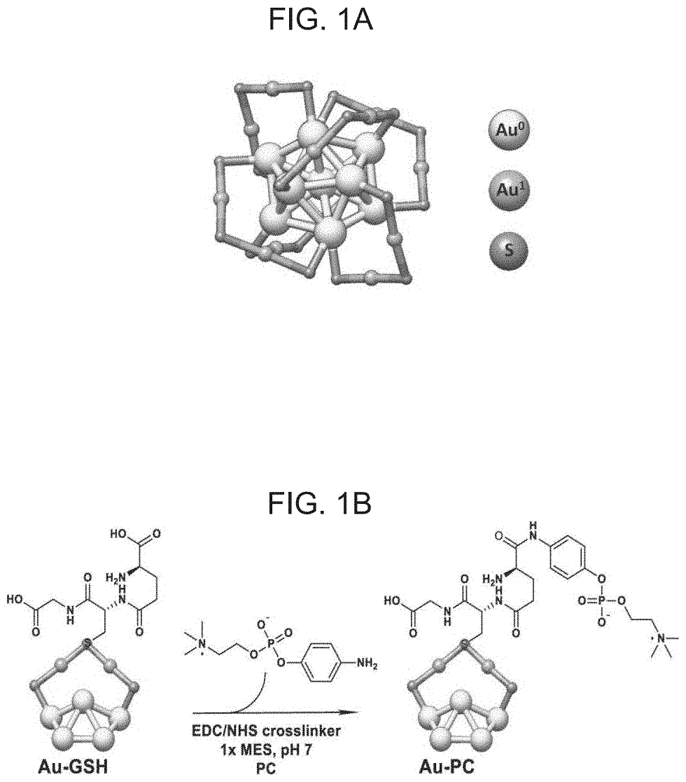

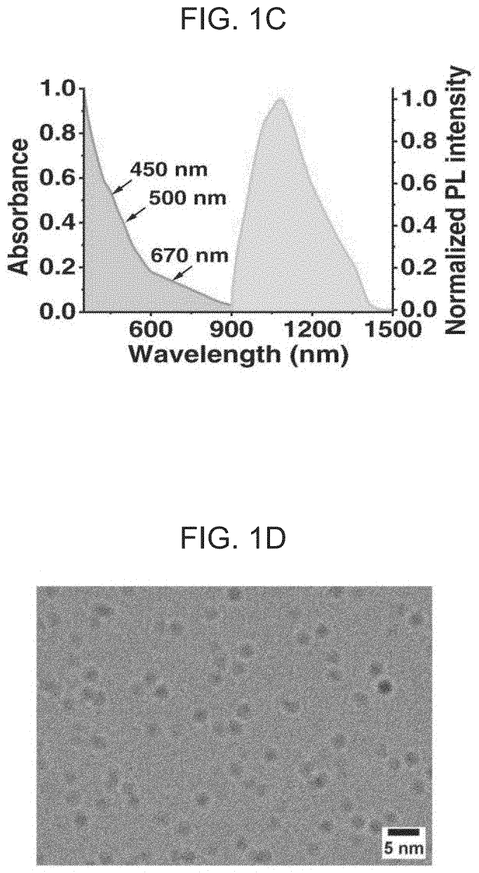

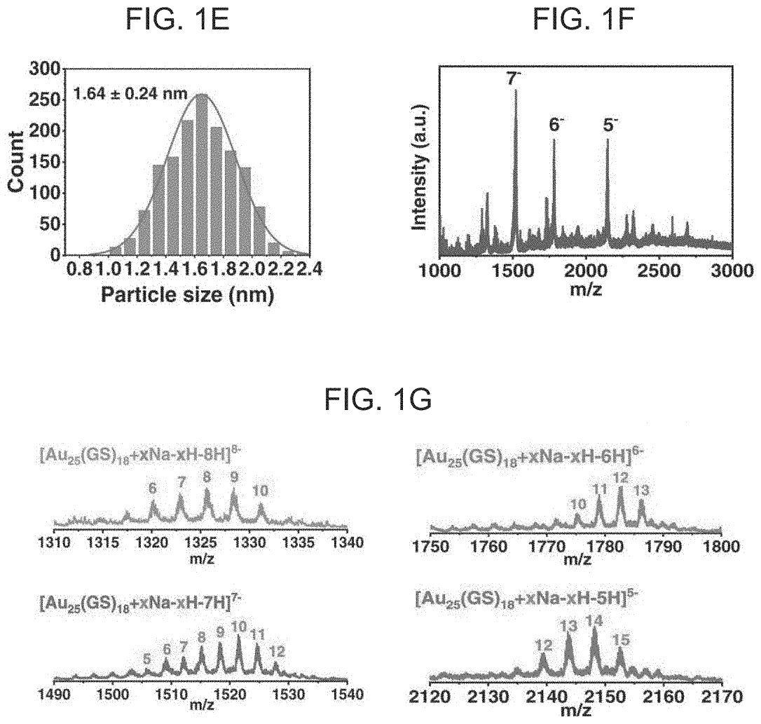

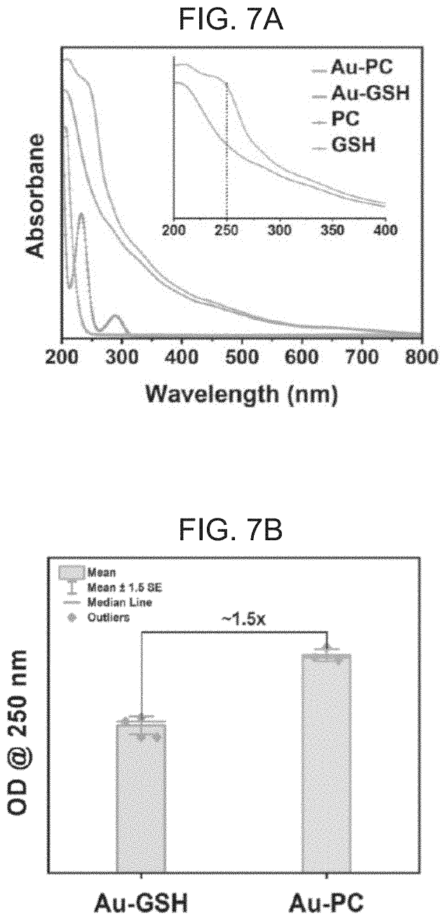

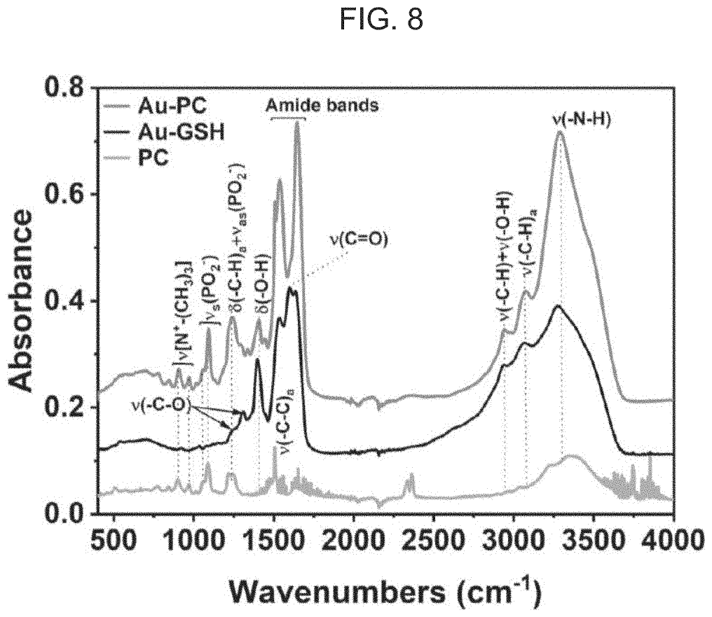

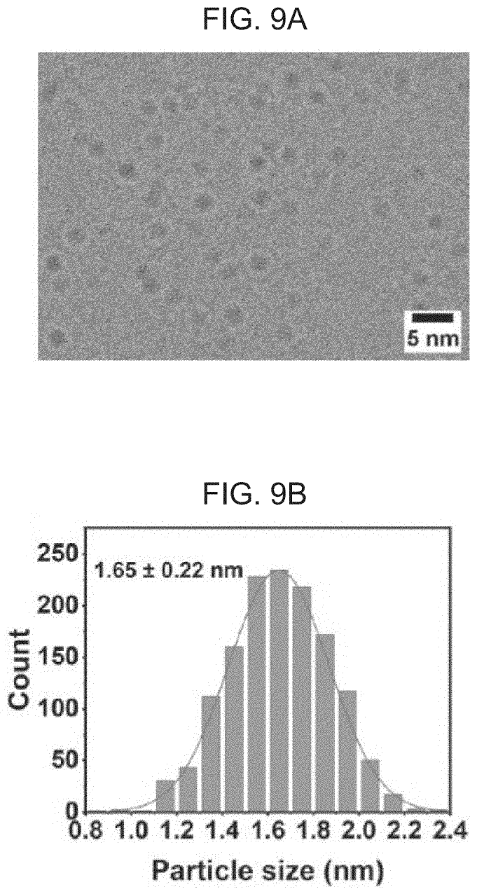

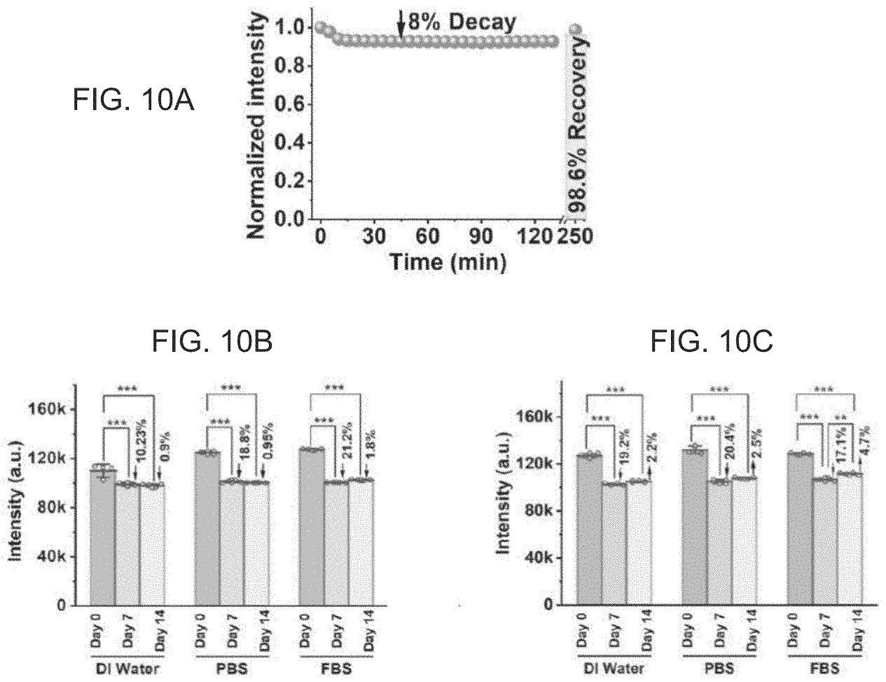

Au-GSH clusters (FIG. 1A) were synthesized in the aqueous phase according to a previously reported method⁶², and then covalently linked the clusters to 4-aminophenylphosphorylcholine (PC) ligands by EDC/NHS chemistry in a MES pH 7.0 buffer followed by purification to afford the Au-GSH-PC conjugates (hereafter referred to as Au-PC, FIG. 1B) (see Methods). The UV-vis absorption of the sample showed a decreasing trend at longer wavelengths typical for Au-SR clusters⁶² (SR: thiol ligand) (FIG. 1C). The conjugation of PC ligand to Au-GSH resulted in the appearance of a bump at 250 nm (associated with PC ligand, FIG. 7A) with a ˜1.5±0.1-fold (or 50%) increase in absorbance and estimated ˜50% conjugation yield (˜18 PC ligands per cluster), FIG. 7B. The conjugation of PC ligand onto the cluster surface has further been validated by the ATR-FTIR spectroscopy (FIG. 8). The characteristic asymmetric (1240 cm⁻¹) and symmetric (1090 cm⁻¹) stretching vibrational modes of PO₂⁻ group and choline headgroup at 970-895 cm⁻¹ can be clearly observed in Au-PC conjugate. The disappearance of the stretching mode at 1600 cm⁻¹ assigned to the presence of sodium carboxylate completely disappeared in Au-PC. The TEM images of Au-GSH cluster (FIG. 1D) and Au-PC conjugate (FIG. 9) obtained from cryo-electron microscope show spherical particles with narrow size distributions with an average size of 1.64±0.24 nm (FIG. 1E) and 1.65±0.22 nm (FIG. 9B), respectively. Under an 808 nm laser excitation, the Au molecular clusters showed photoluminescence (PL) in the NIR-II window with the maximum peak located at ˜1090 nm (FIG. 1C), similar to previous reports^{52,55}. Over 2 h of continuous 808 nm laser irradiation at a power density of 35 mW/cm², stable luminescence was observed over time after an initial ˜8% decay (FIG. 10A). The photoluminescence (PL) stability of Au-GSH cluster and Au-PC in water, PBS and FBS before and after two weeks was studied (FIG. 10B-10C). The PL intensity of Au-GSH and Au-PC increased slightly in PBS and FBS compared to water at Day 0, however, the corresponding intensities were decreased ˜10-21% for Au-GSH and ˜17-19% for Au-PC after a week of incubation. No further decrease in intensity has been observed after two weeks of incubation. The absolute NIR-II emission quantum yields of the Au-GSH and Au-PC clusters excited at 808 nm were measured in the 900-1500 nm emission range to be ˜0.27 and 0.38% respectively using an integrated sphere technique (see Methods).

The gold clusters synthesized were molecular in nature (ultra-small size <3 nm) without plasmonic features, characterized to be Au₂₅-GSH on average (FIG. 1F and 1G), but with a degree of inhomogeneity⁶². Electro-spray ionization (ESI) mass-spectrometry measurement identified several negatively charged species (FIG. 1F) with sodium adducts and were assigned to be [Au₂₅(GS)₁₈+xNa-(x-z)H]^{z-}, where x is the number of sodium adducts, z is the charge (FIG. 1G).

### Example 2—In Vivo NIR-II >1100 nm Fluorescence Imaging with Intravenous, Intra-Tumoral and Subcutaneous Injected Au-PC, Au-GSH and ICG

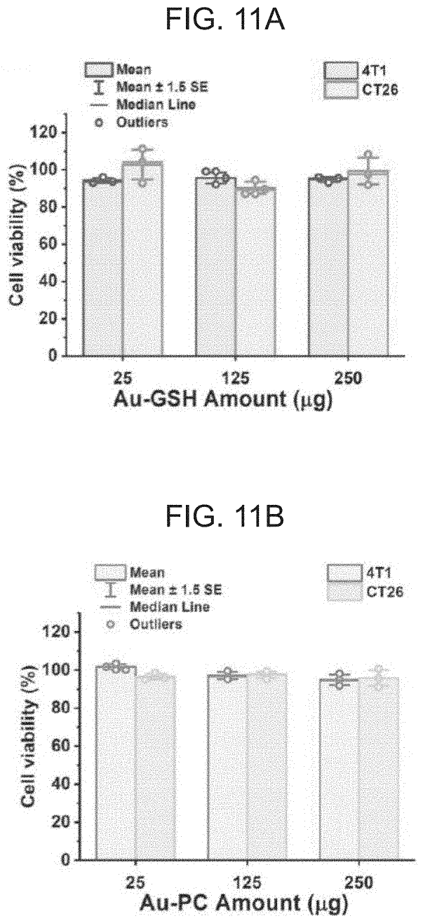

In vitro, no cytotoxicity of the parent Au-GSH and Au-PC clusters was observed when 4T1 murine breast cancer cells and CT26 colon cancer cells were incubated with different mass concentrations of the clusters, at 1 mg/mL, 5 mg/mL and 10 mg/mL concentrations for 12 h at 37° C. (FIG. 11). This is unsurprising since both GSH and PC ligands are naturally abundant biomolecules and gold is a safe element. GSH is involved in antioxidant defense against reactive oxygen species (ROS) activity, nutrient metabolism and in cellular events⁶³ whereas the PC ligand is a component of cell membrane.

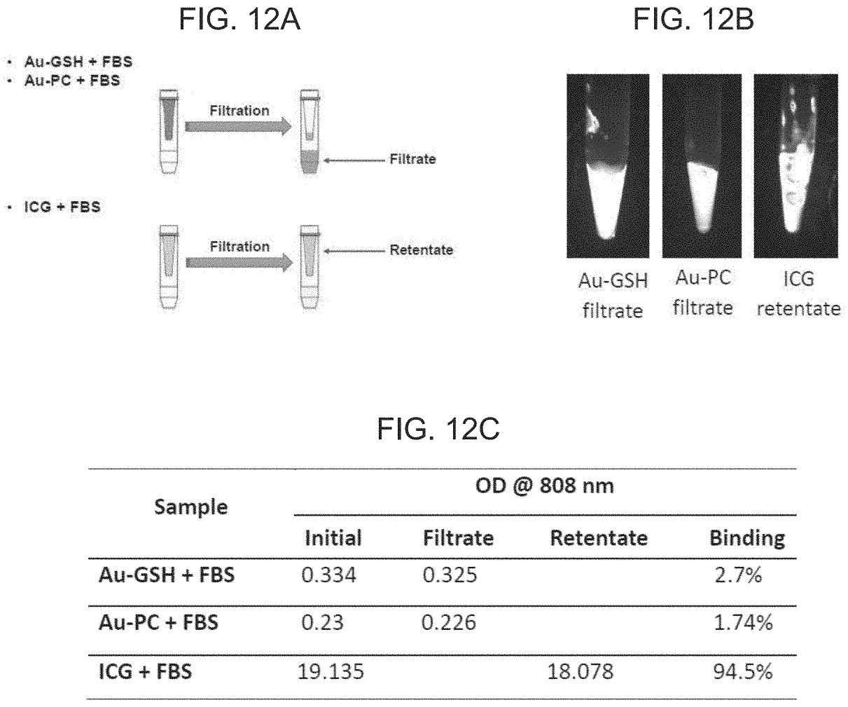

Evaluated next were the serum protein binding capabilities of Au-GSH cluster and Au-PC conjugate with FBS and compared to that of ICG. Briefly, the clusters and ICG were incubated with FBS for 1 h at 37° C. (FIG. 12). Afterwards, the solutions were filtered using Amicon 50 kDa centrifuge filters (FIGS. 12A and 12B). Au clusters bound to serum proteins will not pass filter while free unbound clusters will be lost to the filtrate. The optical density (OD) of filtrate (in case of Au-GSH and Au-PC) and retentate (in case of ICG) at 808 nm were measured and the corresponding serum protein binding efficiencies were calculated (FIG. 12C). The schematics of the experiments, figures, and absorbance values of Au-GSH+FBS and Au-PC+FBS filtrates as well as ICG+FBS retentate can be found in FIG. 12. The serum protein binding efficiencies for Au-GSH cluster, Au-PC conjugate and ICG were calculated to be 2.7%, 1.74% and 94.5%, respectively. That is, most 94.5% ICG was found to bind to serum protein and failed to pass through the filter. While both Au-GSH and Au-PC showed much lower interaction with serum proteins, especially Au-PC.

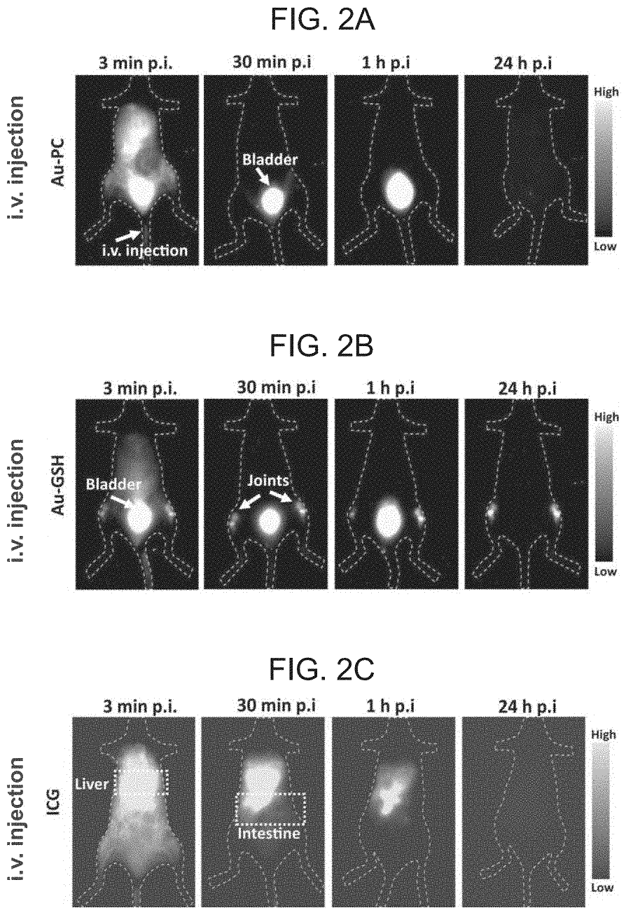

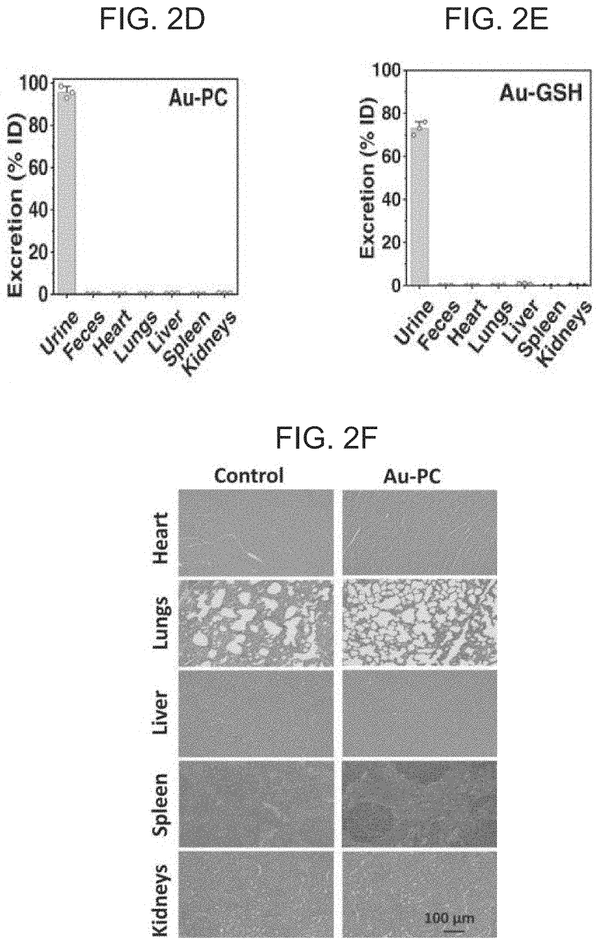

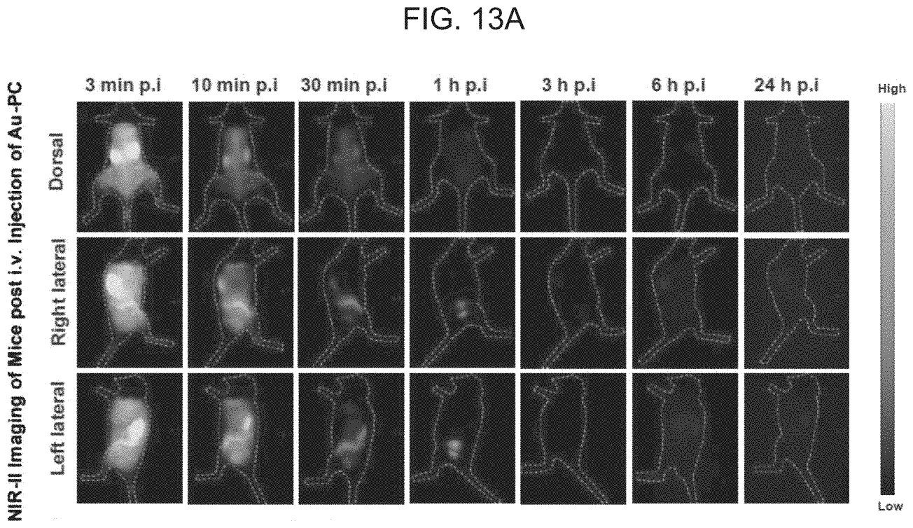

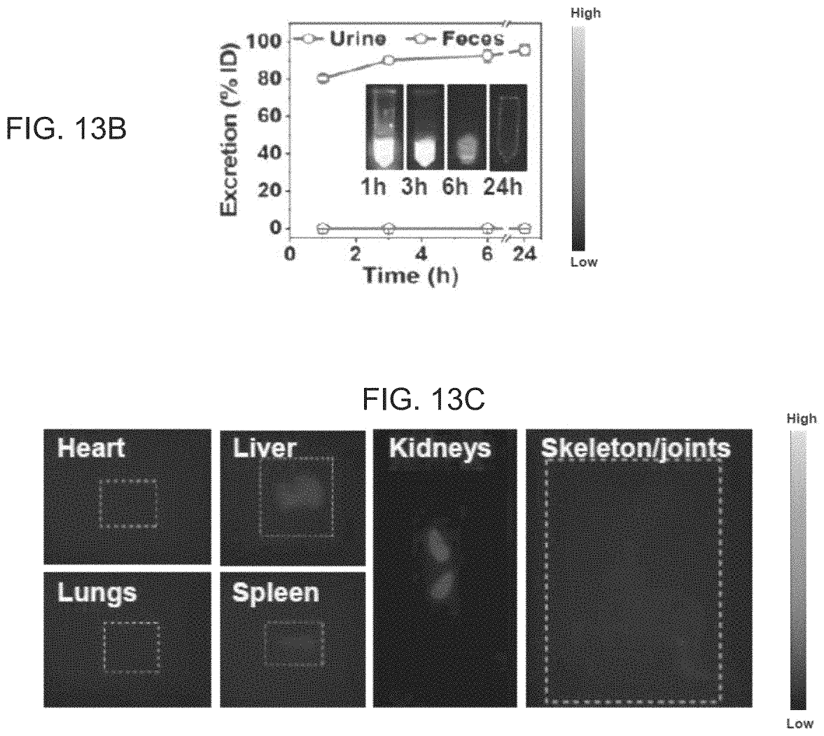

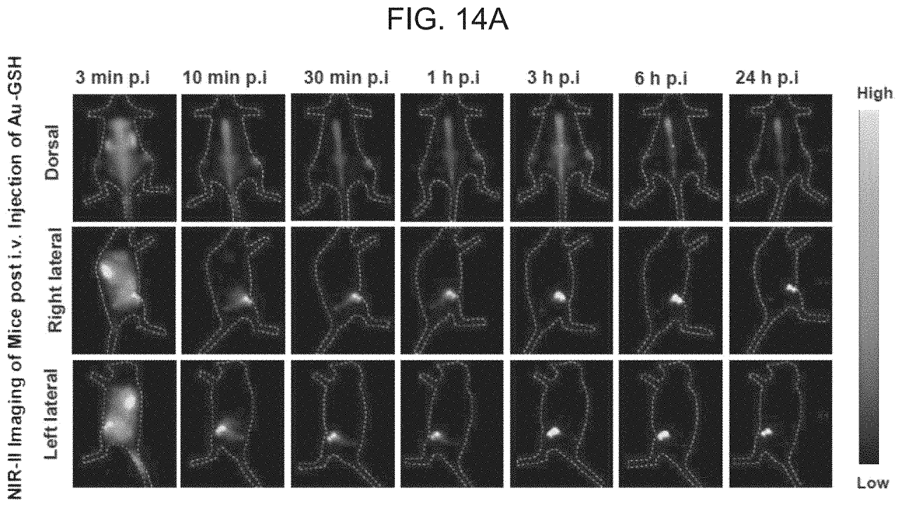

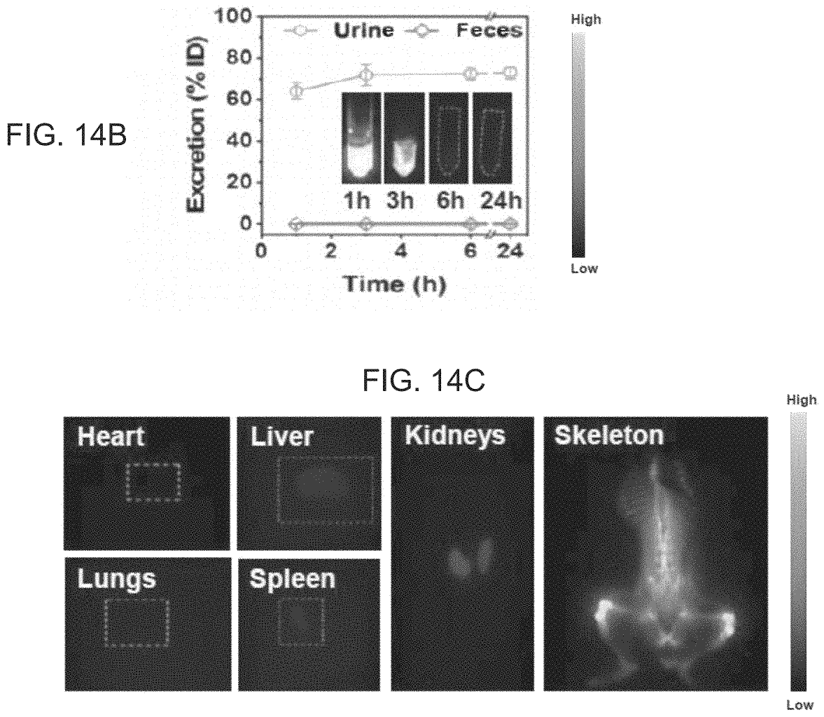

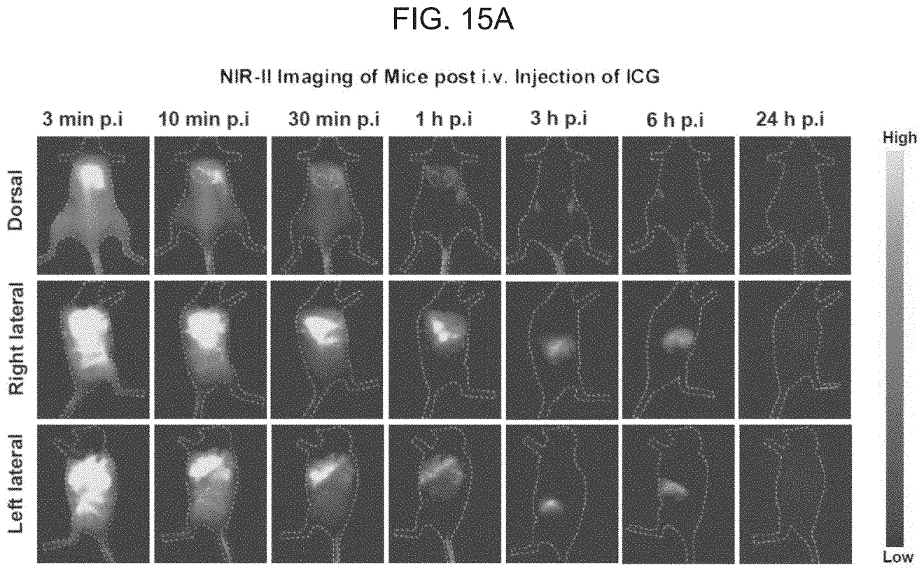

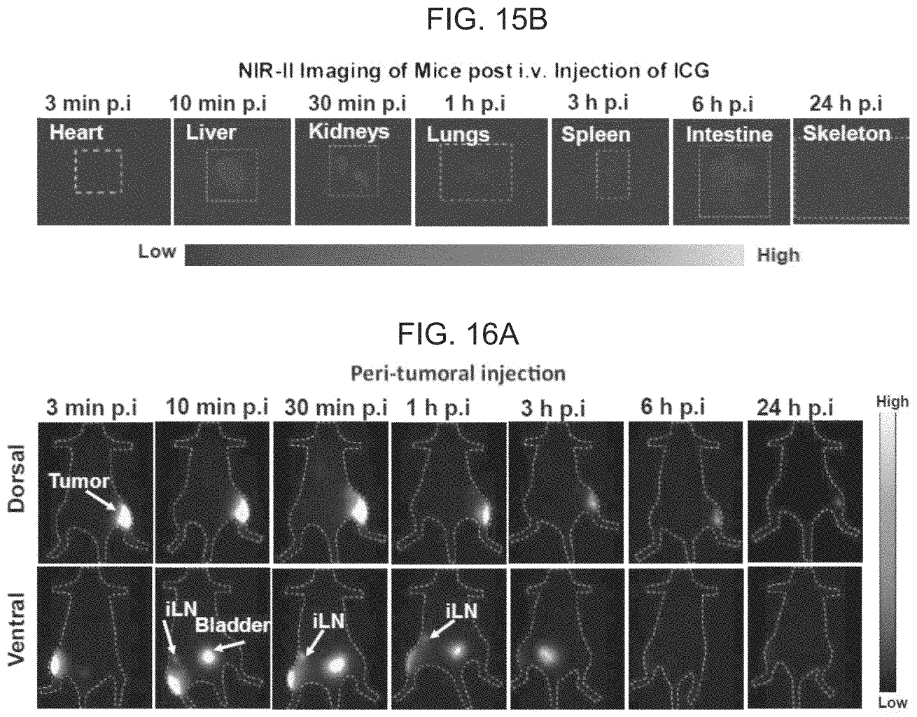

In vivo, the Au-GSH and Au-PC clusters dissolved in PBS were first intravenously (i.v.) administered to mice (5-7 weeks old female Balb/c, n=3 in each group) through tail-vein injection, imaged in the >1100 nm NIR-II window and compared side by side with the clinically approved ICG dye. The bladder NIR-II signals in mice rapidly lit up, at ˜3 min postinjection (p.i) (FIG. 2A, 2B ventral view), as a consequence of kidney drainage for fast renal clearance (dorsal/lateral view images in FIGS. 13A and 14A, respectively). ICG was shown to exhibit a fluorescence tail extending to the >1000 nm NIR-II window⁶⁴. For intravenously injected ICG, strong NIR-II signals were observed in liver and intestine, consistent with the biliary excretion route in the form of ICG-serum protein binding complexes⁶⁵ (FIG. 2C). The body signal was cleared out after 24 h postinjection with no significant ICG retention in major organs (FIG. 15).

The intravenously administrated Au-GSH clusters showed a degree of non-specific accumulation/retention in the central skeletal system (spinal cord and joints in particular, FIG. 14C)⁵⁵. ICP-MS analyses showed ˜64% of i.v. injected Au-GSH was excreted with urine within 1 h p.i. and reached a total of ˜73% excretion in one day with about 1% of gold remained in the liver and 0.35% in the kidneys (FIG. 2E and FIG. 14B). For Au-PC, such bone signals were no longer observed and the Au-PC freely excreted with urine without retention in major organs, suggesting a highly stealth nature of the Au-PC clusters (FIG. 13C) without binding to serum proteins. Observed was ˜81% of Au-PC excreted in the urine within 1 h p.i. and further increased to ˜93% at 24 h p.i. (FIG. 2D and FIG. 13B). The Au-PC clusters are highly stealth with little non-specific interactions with proteins and other biological species likely contributed by two factors previously elucidated for alkylthiol-PC monolayers on gold by experiment and simulations⁶⁶. The first is strong water hydration of the zwitterionic PC group by water molecules through electrostatic forces, and the second is minimal net dipole moments of PC head groups oriented anti-parallelly nearly normal to the Au surface⁶⁶. Both factors likely contributed to minimal non-specific interactions between Au-PC and proteins.

Histological sections of hematoxylin and eosin (H&E)-stained major organs from untreated mouse and a mouse injected with Au-PC conjugate show no differences (FIG. 2F), suggesting high safety of intravenously injected Au-PC probes in vivo.

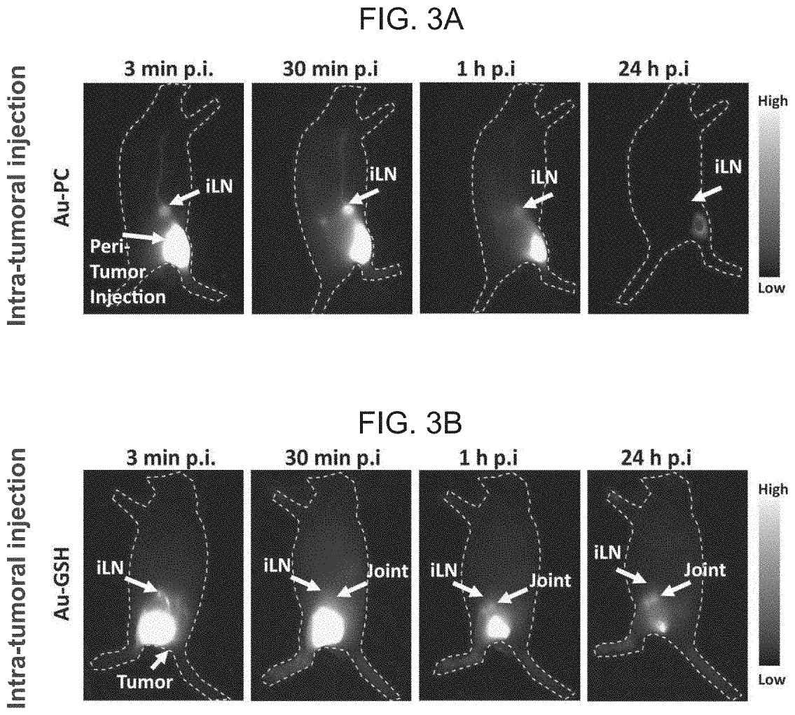

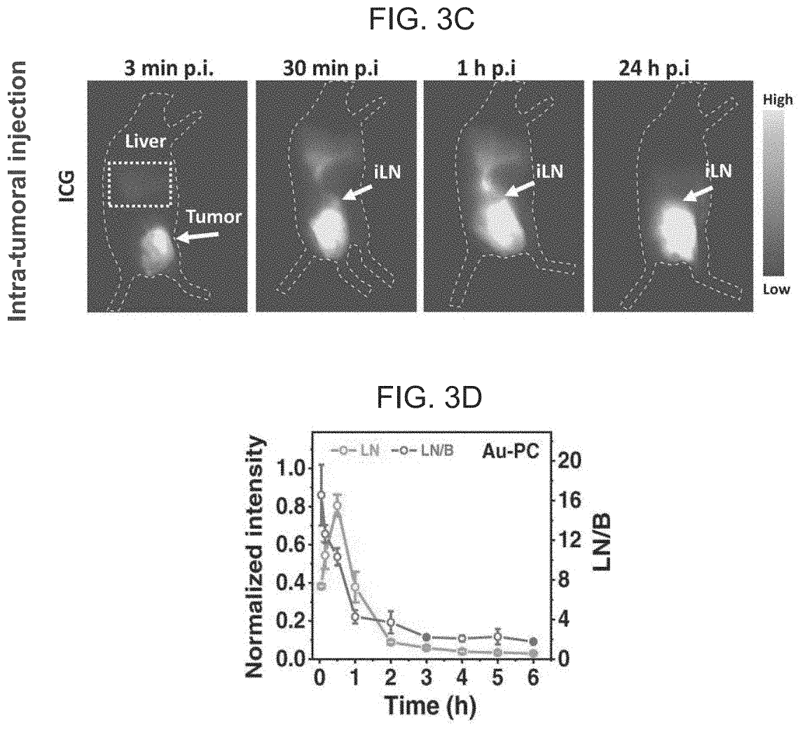

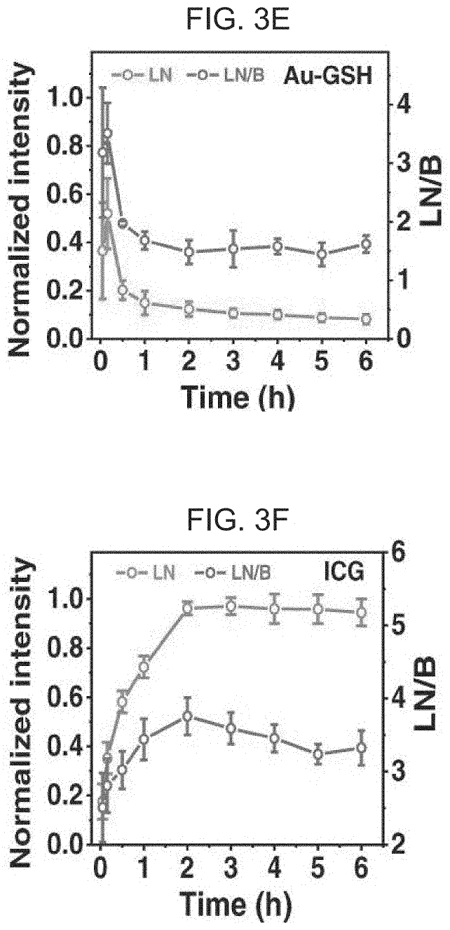

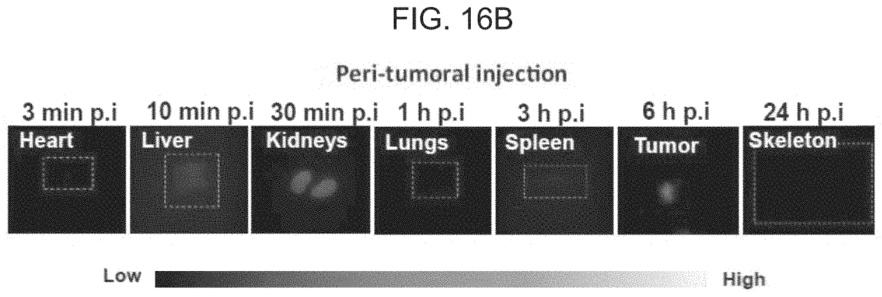

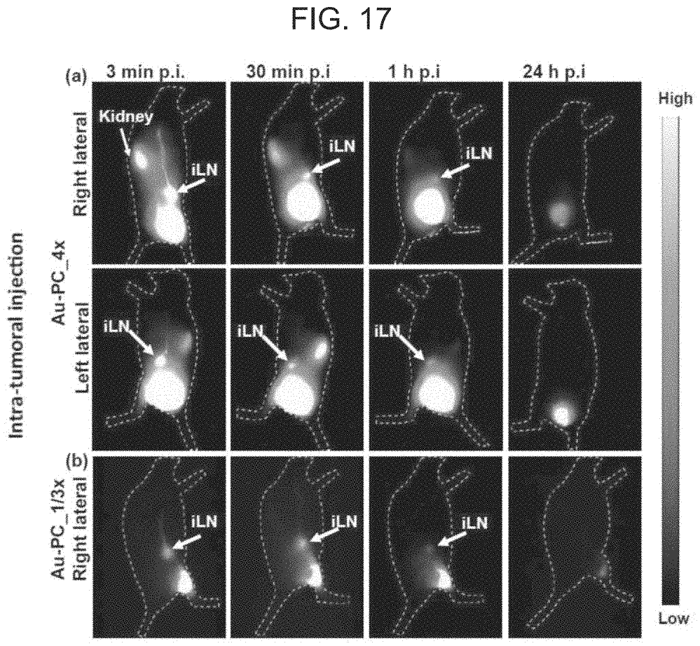

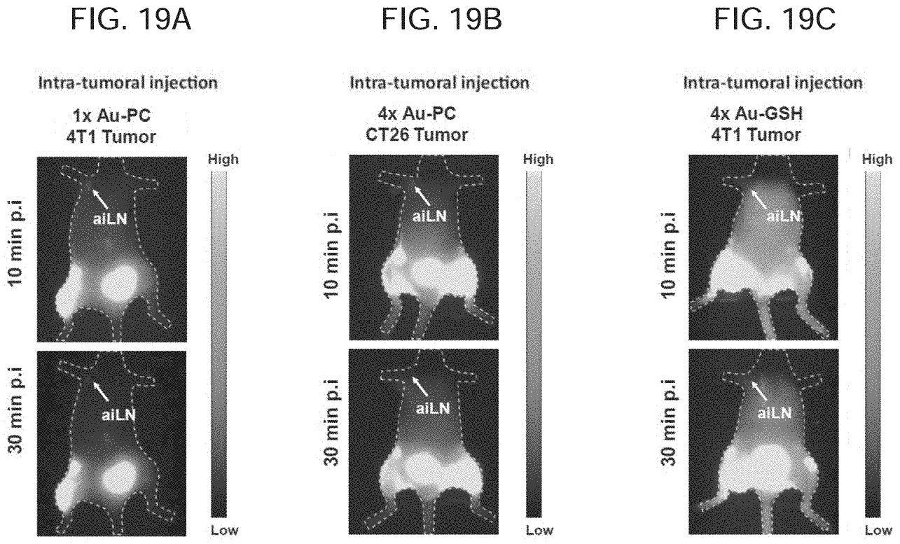

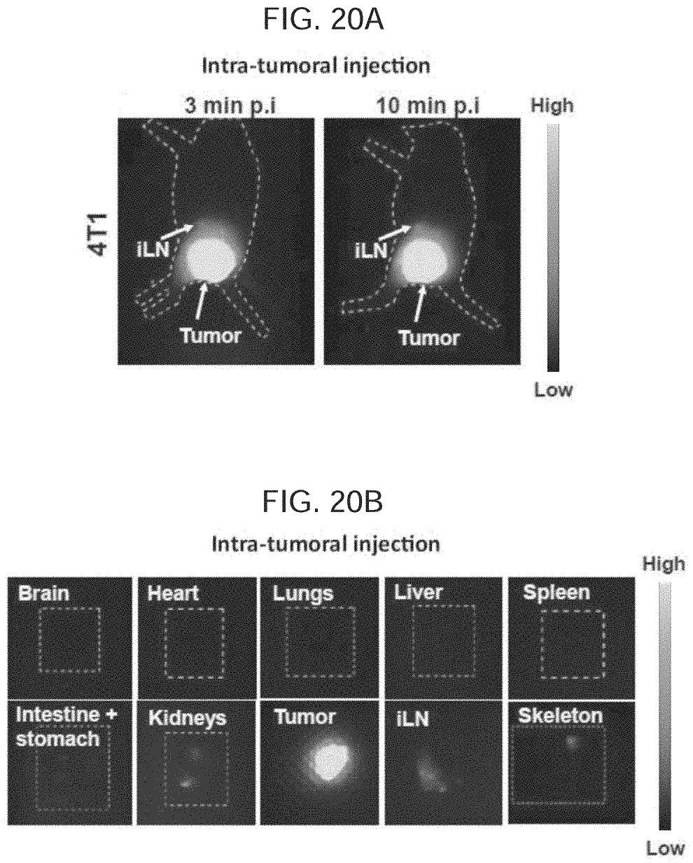

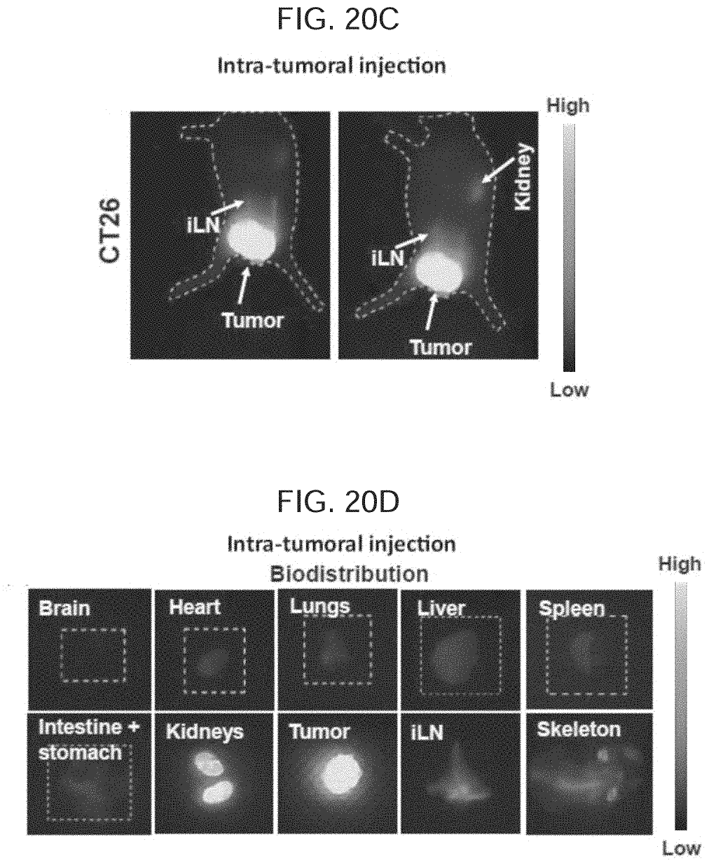

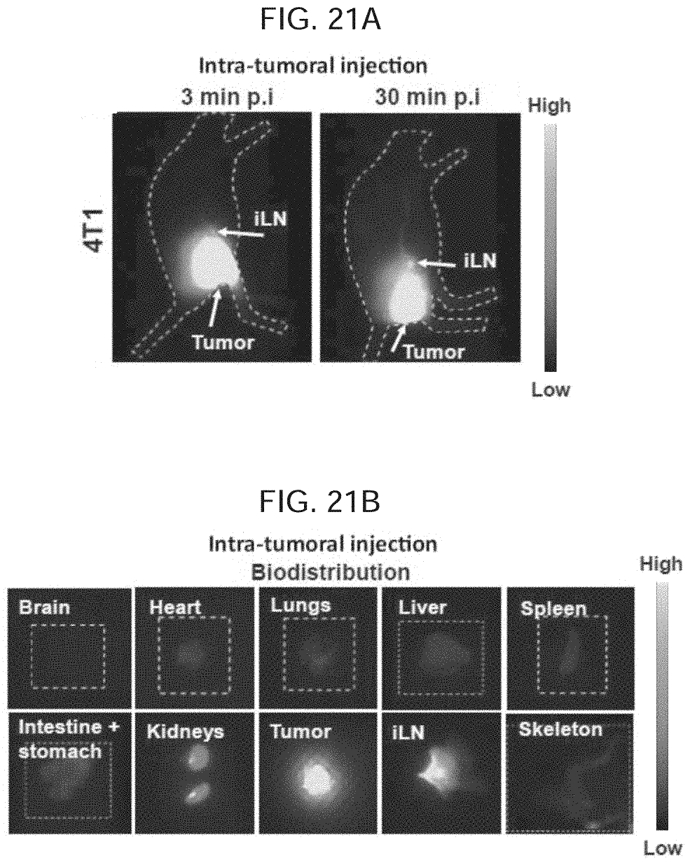

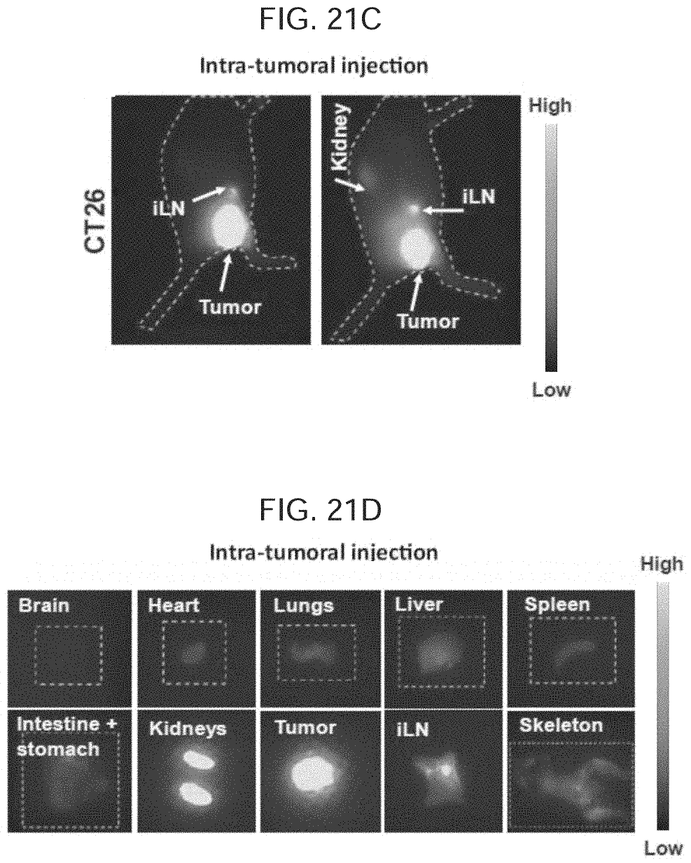

For lymph node imaging, intra-tumor/peri-tumor administration (i.t.) of Au-PC clusters to mice (5-7 weeks old female Balb/c, n=3 in each group) bearing syngeneic 4T1 murine breast tumors and CT26 colon tumors inoculated on hindlimbs was performed. Several doses of Au-PC probes, including 4× (˜1.2 mg, tumors on both hindlimbs), 1× (˜300 μg, tumor on the right hindlimb) and ⅓× (˜100 μg, tumor on the right hindlimb) were administered (FIG. 3A, FIGS. 16 and 17). The draining inguinal lymph nodes (iLN) started to show NIR-II emission of Au-PC within ˜1 min p.i. and reached high brightness within ˜3 min p.i. (FIG. 3A, FIG. 17 for 4T1; FIG. 12A for CT26 tumor). The LN signal persisted for over 1 h in the draining iLNs (FIG. 3A) after reaching peak intensity at ˜30 min p.i., with a LN/background signal ratio ˜5-10 (FIG. 3D, FIG. 12D). In 10 min p.i., Au-PC NIR-II emission in the lymphatic vessel from iLNs reaching up to the axillary region and weakly labeled the axillary LN (aLN) for both 4T1 and CT26 mouse models was observed (FIG. 19A and 19B). The signal completely disappeared after 30 min p.i. of the Au-PC probe. The Au-PC clusters afforded rapid and effective imaging/detection of the primary tier draining LNs with much weaker signals in higher tier nodes.

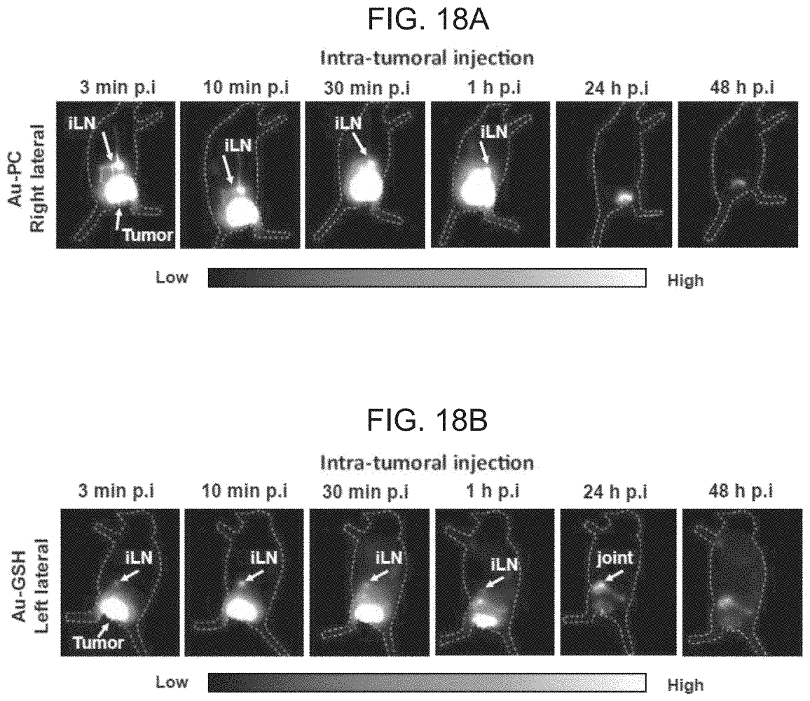

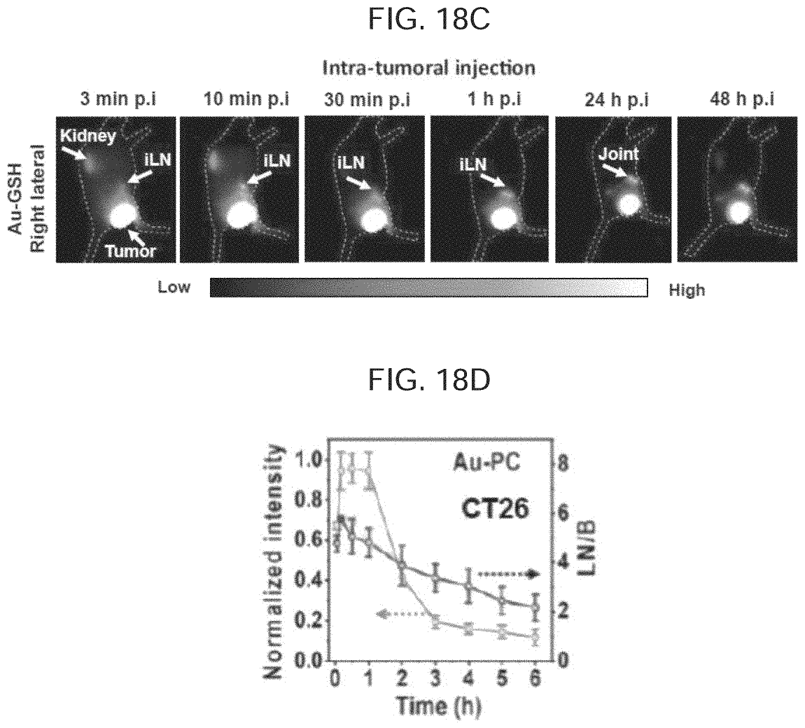

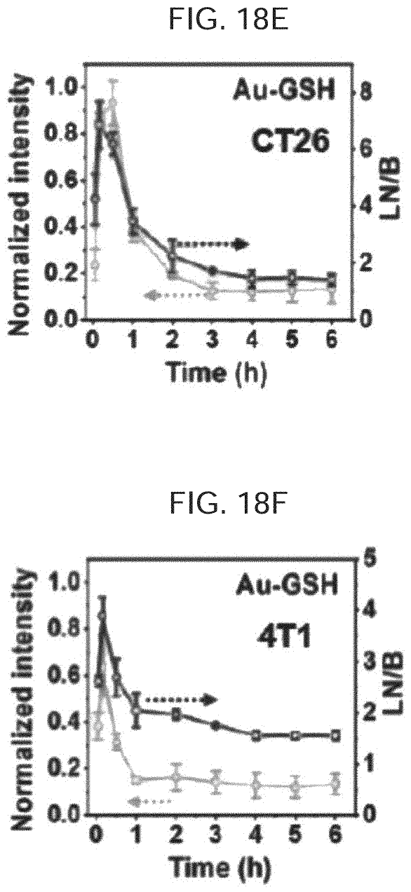

To compare with Au-PC, i.t. administration of Au-GSH probes into 4T1 tumors (5-7 weeks old female Balb/c, n=3 in each group) (FIG. 3B and FIG. 18C right lateral view) and CT26 tumors (FIG. 18B) was performed, and a much shorter time span for the LN draining process was observed. The NIR-II signals detected in LNs upon 1× (FIG. 3B, 3E) and 4× (FIG. 18B and 18C) dosage administration of Au-GSH were relatively low and not significantly different in intensity. The signals in the iLNs reached its maximum intensity rapidly after ˜10 min p.i. with LN/background (LN/B) signal ratios of ˜2-4 (FIG. 3E and FIG. 18F) for 4T1 tumors and ˜6-7 (FIG. 18E) for CT26 tumors respectively, and quickly cleared out from the lymphatic system. Similarly, very weak aLNs were detected (FIG. 19C). These results suggested Au-GSH as a less ideal LN imaging agent than Au-PC since the latter lighted up the LN much more brightly over a longer time scale of ˜1 h post-injection.

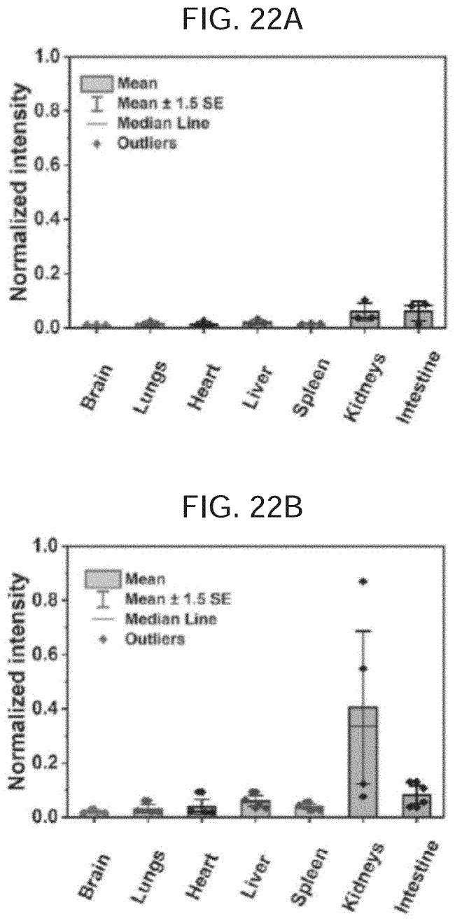

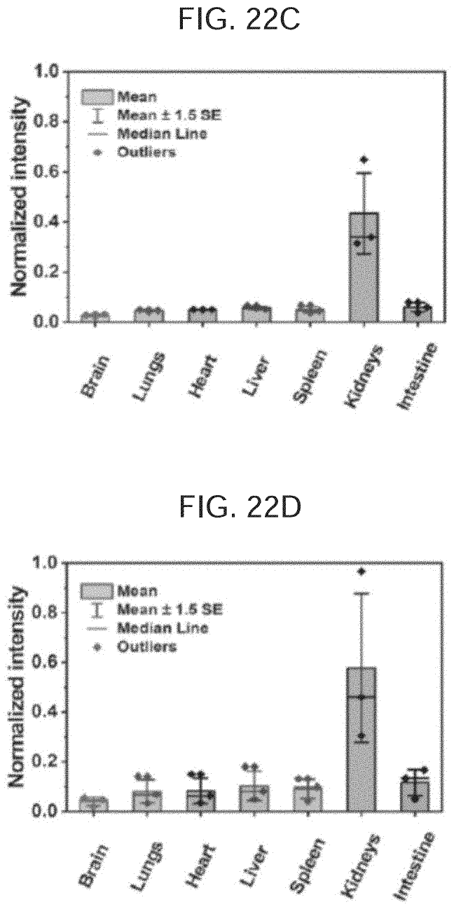

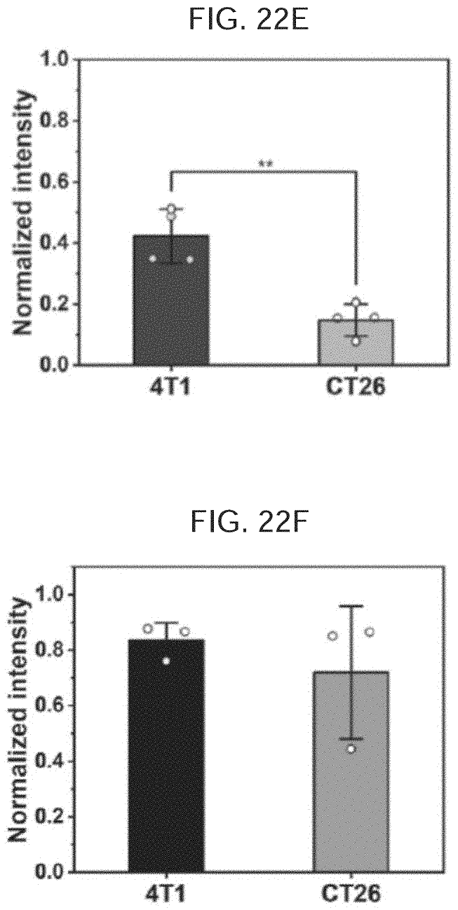

Videos were obtained showing NIR-II fluorescent signals in major organs at the highest lymph node draining time point, i.e., 10 min p.i. for Au-GSH and 30 min p.i. for Au-PC, after intratumoral administration of 1× Au-GSH and Au-PC probes. Compared to NIR-II signals in iLNs 10 min post-administration of Au-GSH probe, ˜3-5-fold higher LN signals were observed after 30 min p.i of Au-PC conjugate (FIG. 22).

Comparing to Au-PC and Au-GSH probes, observed was that ICG exhibited a drastically different SLN draining kinetics (FIG. 3C, in situ probe administration and real-time NIR-II in vivo imaging of draining lymph node, 5-7 weeks old female Balb/c, n=3). The times ICG signal first appeared in the SLN were longer than those of Au-PC and Au-GSH and varied from mouse to mouse, in the range of 10 min-30 min post i.t. injection into 4T1 tumor. ICG^{6,7} is known to bind to serum proteins, slowing down the kinetics of SLN draining. The signal in the lymph node increased gradually and reached peak intensity at 2-3 h post i.t. injection with LN/B ratios of —4 (FIG. 3F). This was in accordance with clinical studies that found the timing for ICG fluorescence signal showing in the sentinel lymph nodes of certain cancers (e.g., oral cancer) was variable, causing uncertainty in the timing between injection and imaging/surgery, ranging from 15 min to up to 24 h^{6,16}. Imaging over time in some cases observed higher tier lymph nodes in addition to the first tier dLN. In this regards, Au-PC clusters differed significantly with little interaction with proteins and cells, transporting through the lymphatics unimpeded and allowing dLN imaging in the comfortably wide minutes to ˜1 h time window postinjection with little timing uncertainty.

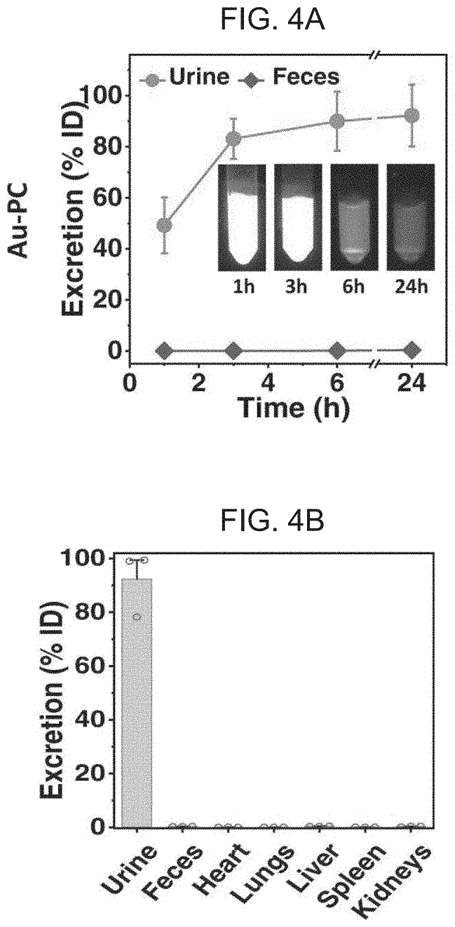

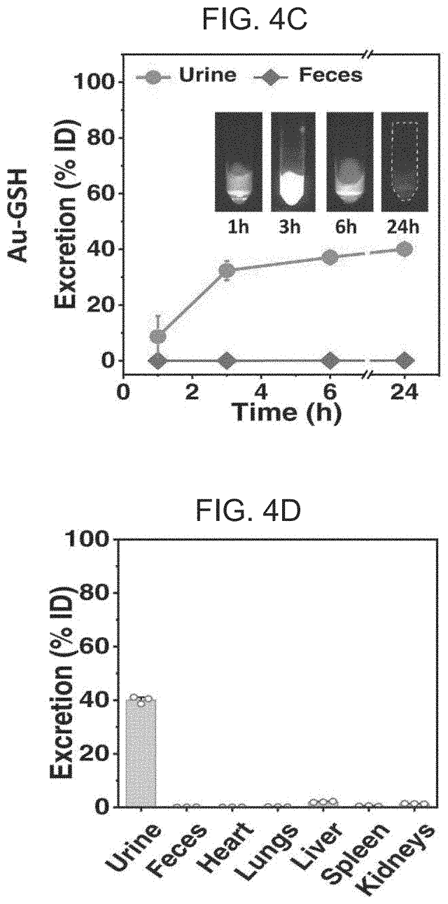

Similar to the intravenous injection cases, the intra-tumoral injected Au-PC and Au-GSH probes excreted out from the body via renal route (FIG. 4), whereas ICG was eliminated through the liver excretory system. Within 24 h, ICP-MS analysis of the excreta showed that about 92% of the injected Au-PC sample excreted via urine (FIG. 4A, 4B) while only 38% urine excretion was observed with Au-GSH (FIG. 4C, 4D). Near complete signal fading from the tumor injection site and from the mouse body was observed 24 h p.i for the Au-PC probe (FIG. 3A), and at the same time significant signals were still detected at the tumor injection sites for Au-GSH (FIG. 3B) and ICG probes (FIG. 3C). The Au-PC clusters exhibited the least trapping and retention at the injection site and in the body compared to ICG and Au-GSH, suggesting the highly stealth nature of the Au clusters owed to the surface phosphocholine ligands imparting minimum interactions and non-specific binding with proteins, cells, and tissues/organs in the body.

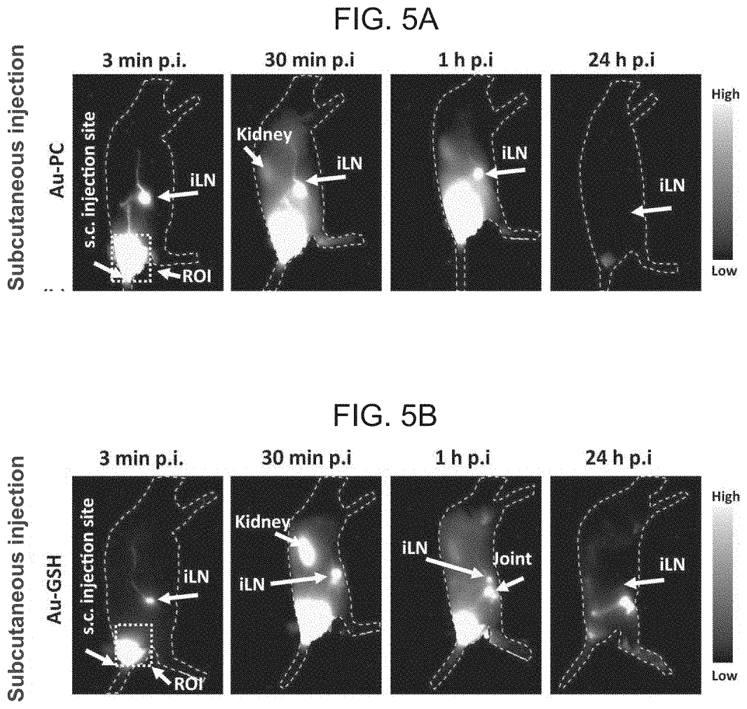

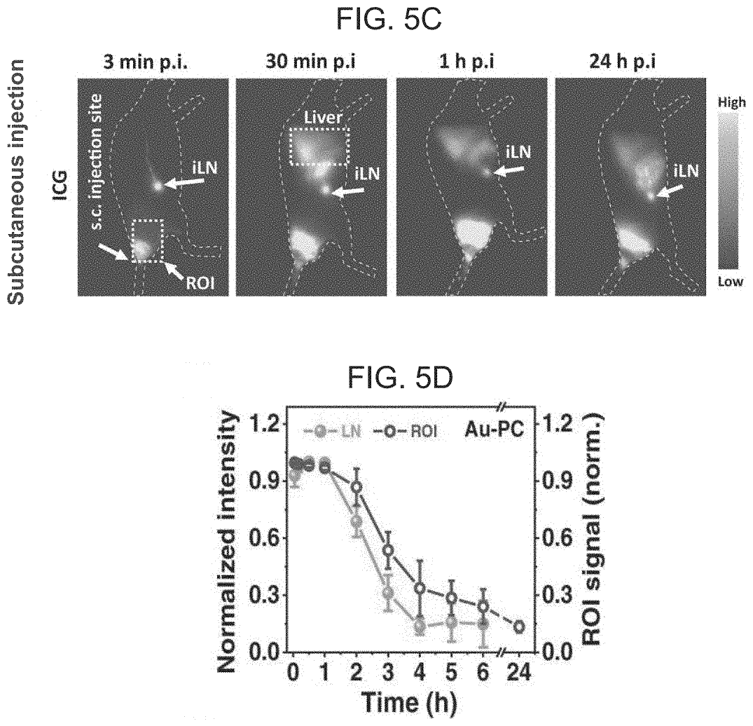

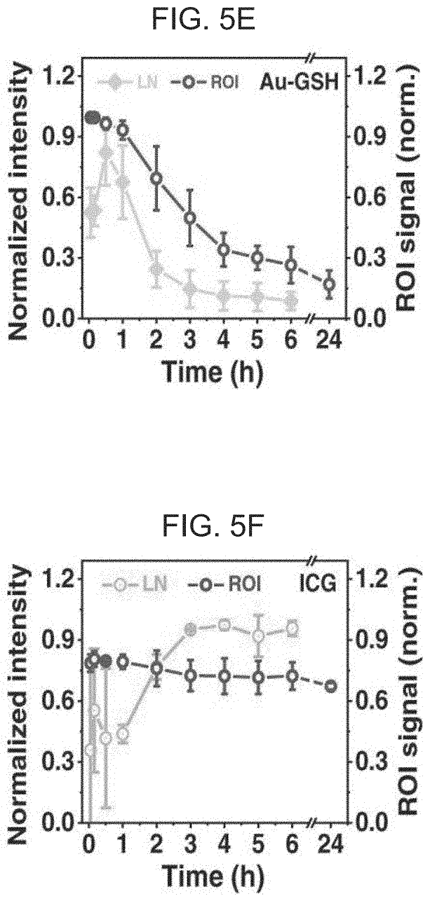

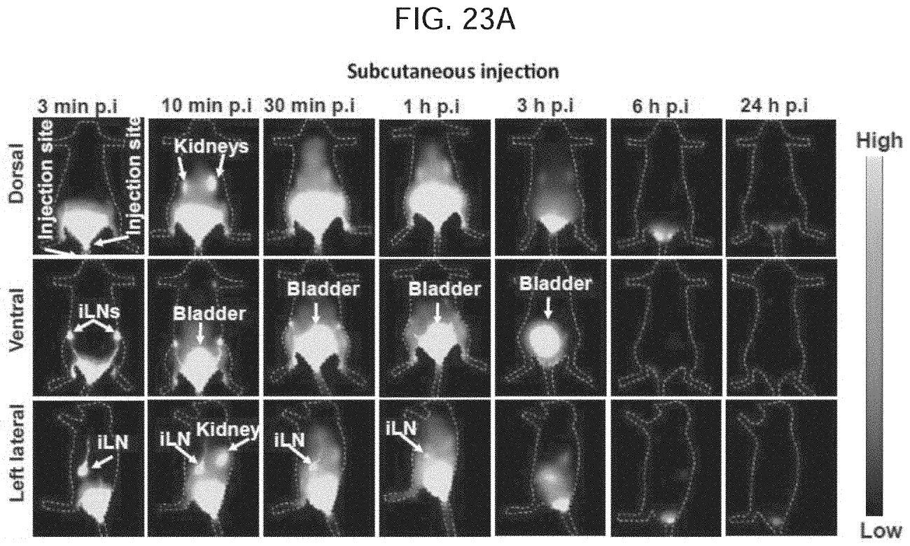

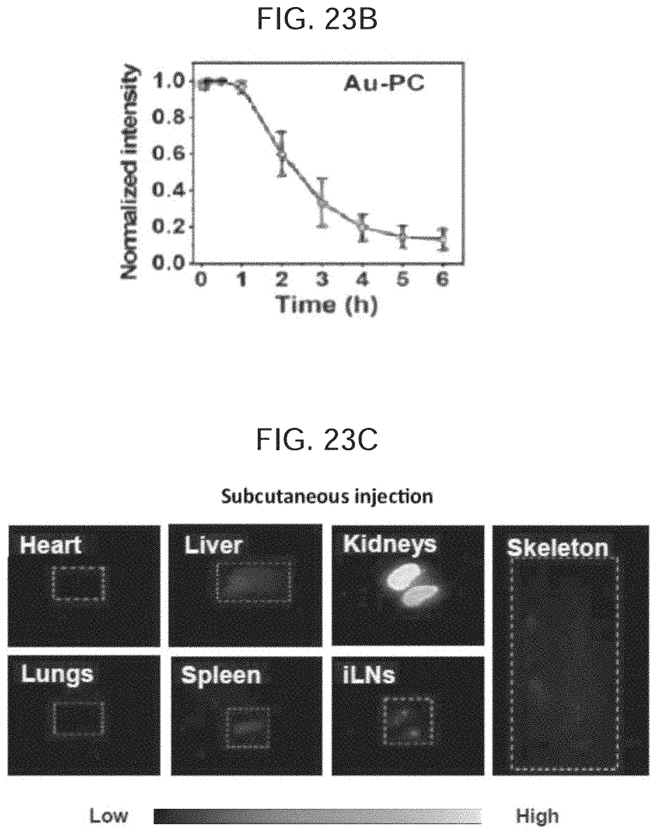

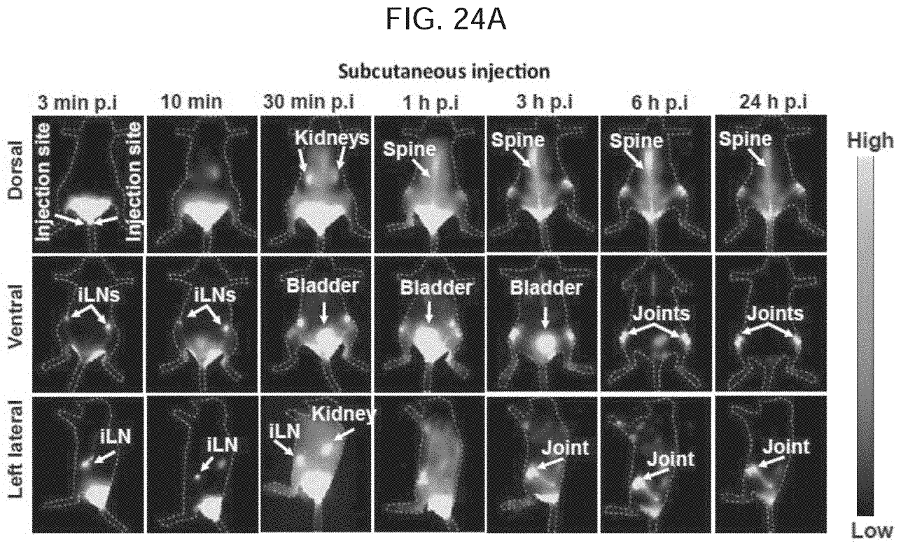

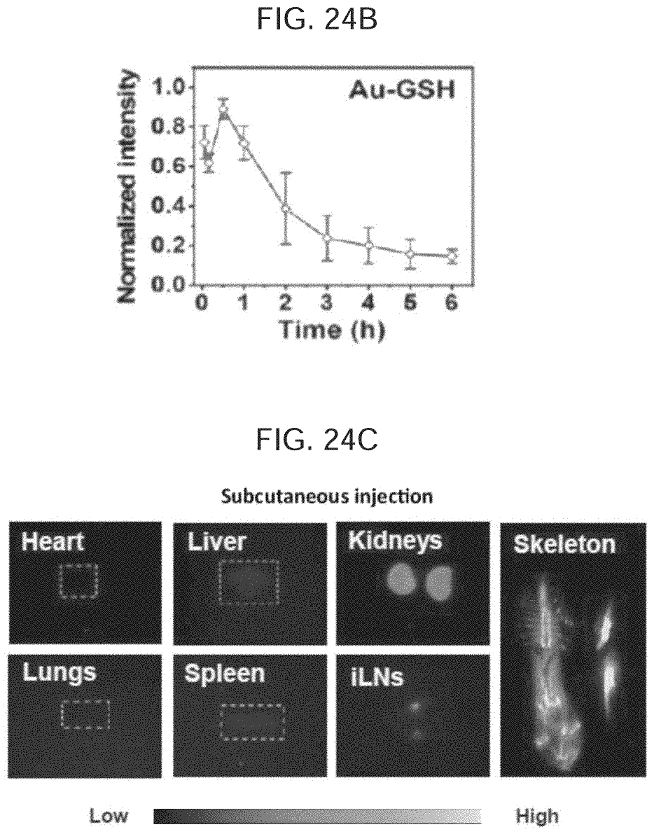

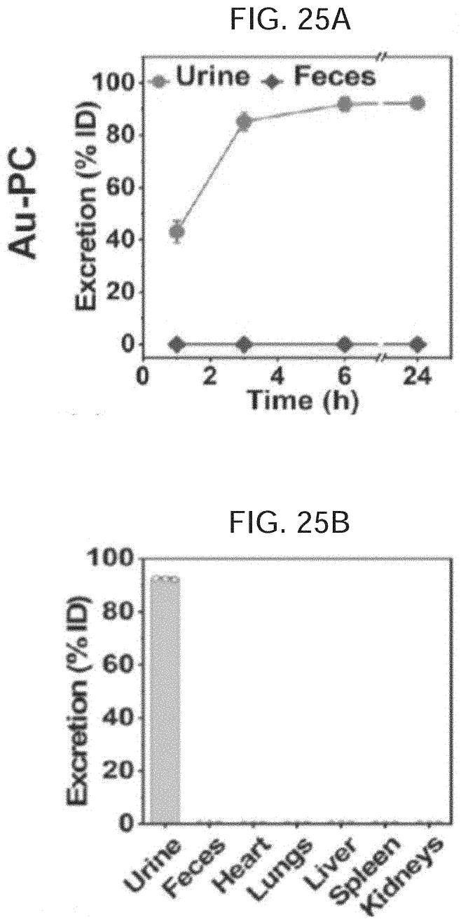

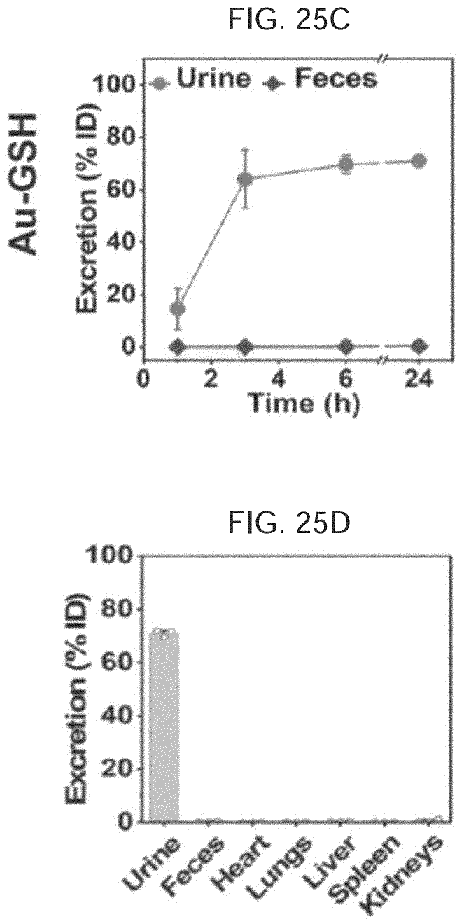

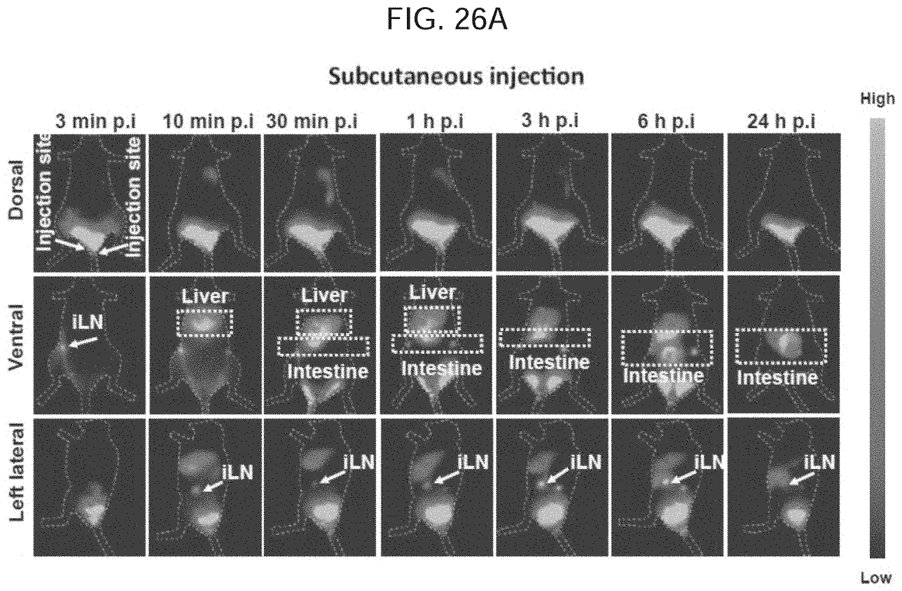

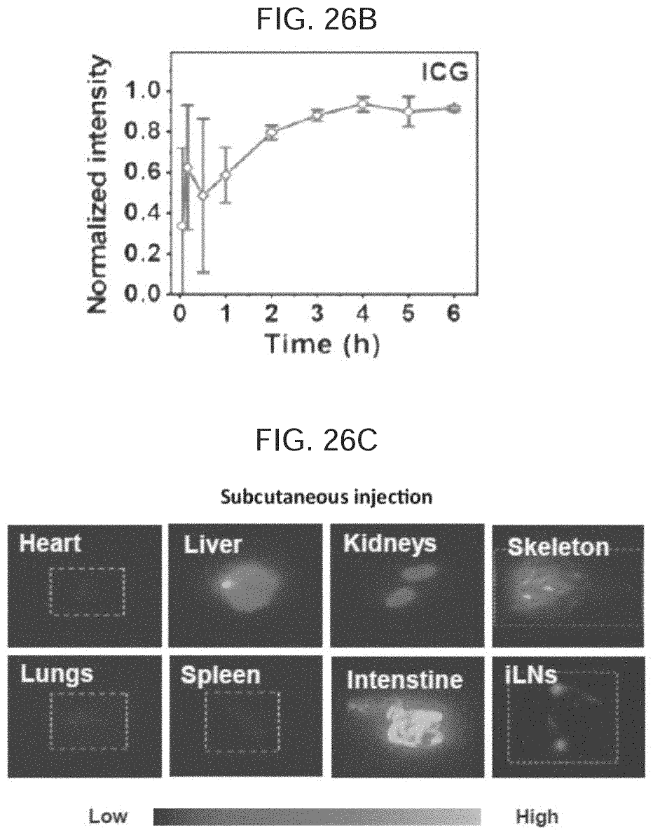

Investigated next was LN draining post subcutaneous (s.c.) injection of the three probes at the mouse tail base (FIG. 5) (5-7 weeks old female Balb/c, n=3 in each group). The Au-PC clusters within 3 min p.i. migrated to the lymphatic vessels connecting the injection site to the draining iLN (FIG. 5A and FIG. 23). Strong signals in the iLN were seen up to 1 h and decreased by ˜40% 2 h p.i. (FIG. 5D). In 24 h, Au-PC signals vanished from the body with little retention at the injection site (FIG. 23). In the case of s.c. injected Au-GSH clusters, signal in the iLN peaked at 30 min, decreased by ˜50% 2 h p.i., and at 24 h mostly vanished from the injection site, but significant signal was observed in the central skeletal framework (FIG. 5B, 5E and FIG. 24). Within 24 h, ICP-MS analysis showed that ˜92% of the injected Au-PC sample was excreted via urine (FIGS. 25A and 25B) while ˜71% urine excretion was observed with Au-GSH (FIGS. 25C and 25D). In contrast, upon ICG administration (FIG. 5C), the fluorescent signal in iLN appeared 3 min p.i. but with an intensity much lower compared to that of Au-PC probe at the same time postinjection (FIG. 5F). After 2-3 h p.i. (or even later) ICG signal in the iLN reached peak intensity and persisted. Even after 24 h p.i. significant signal still remained in the lymph node, at the injection site and in the liver (FIG. 5F and FIG. 26). The retention of ICG at the injection site persisted over an extended period of time (FIG. 5F) much longer than Au-PC. The Au-PC molecular clusters were also unique with little retention at subcutaneous injection sites among various nanomaterials (with well-coated hydrophilic layers such as PEG) including quantum dots (QDs), carbon nanotubes (CNTs) and organic NIR II dyes^{67,68}. The trapping of NIR-II probes at the injection site and staining it for weeks is undesirable due to potential long-term side effects.

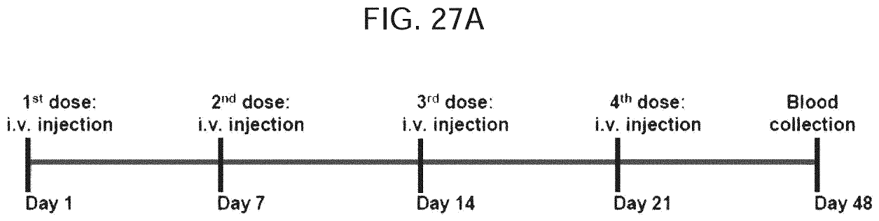

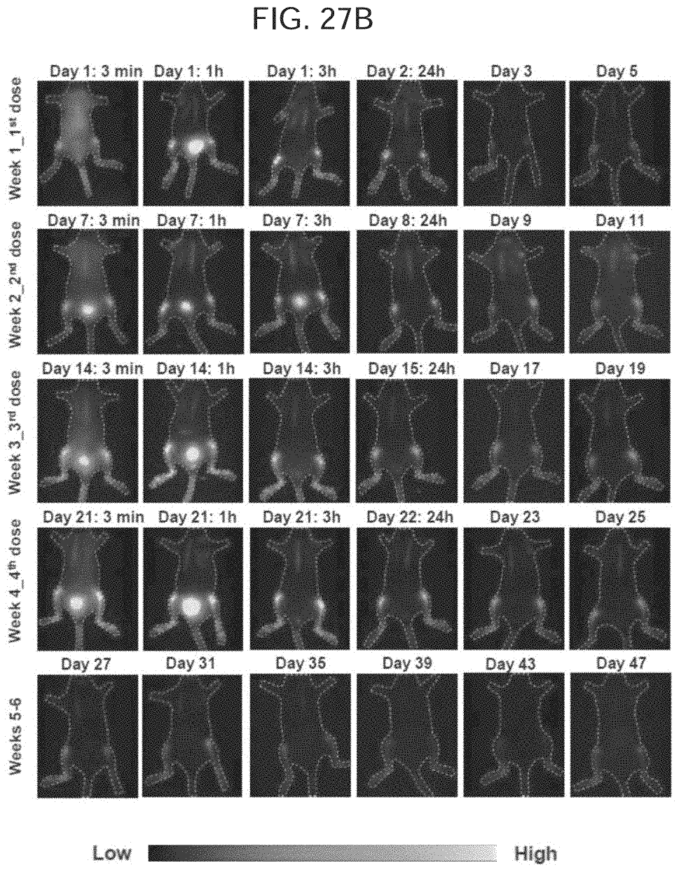

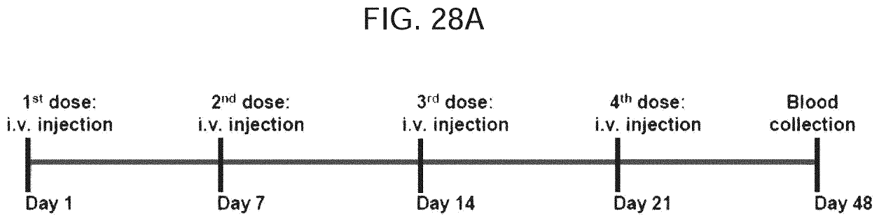

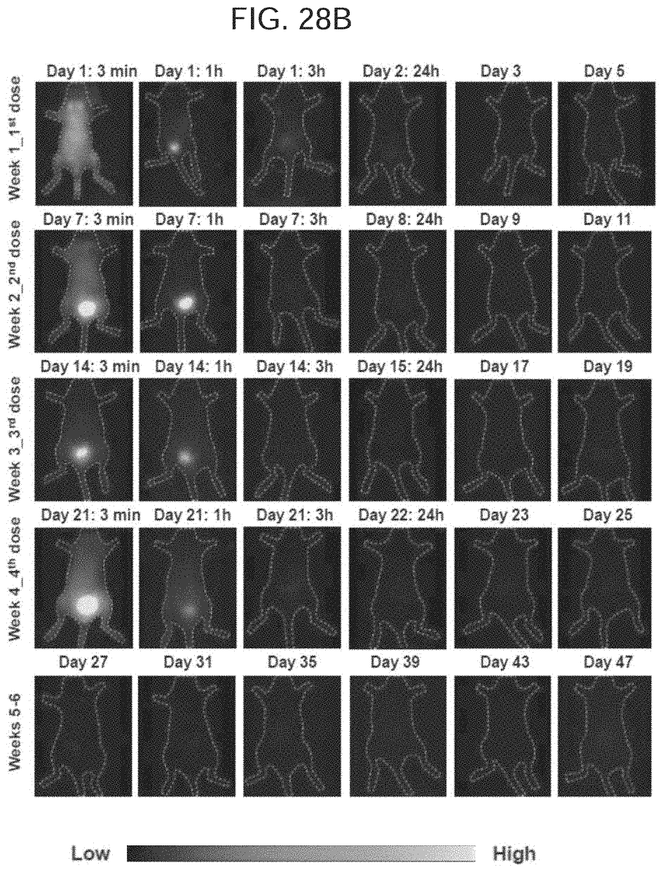

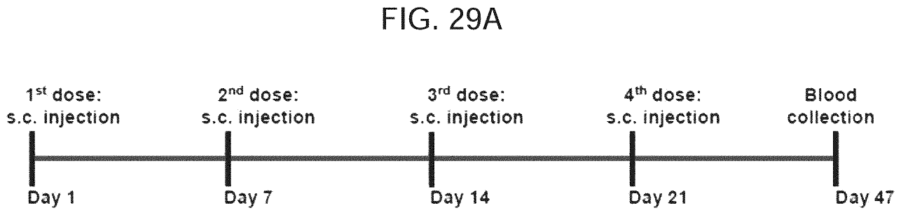

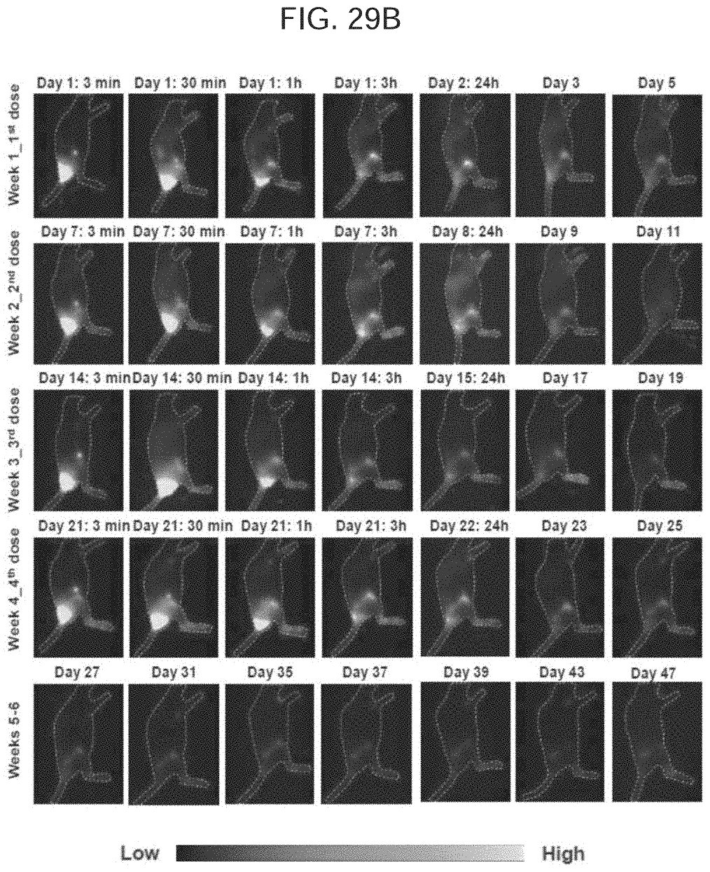

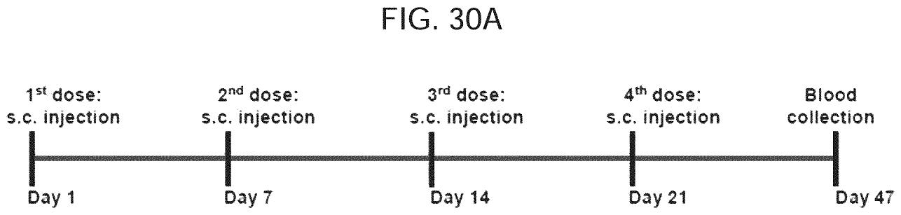

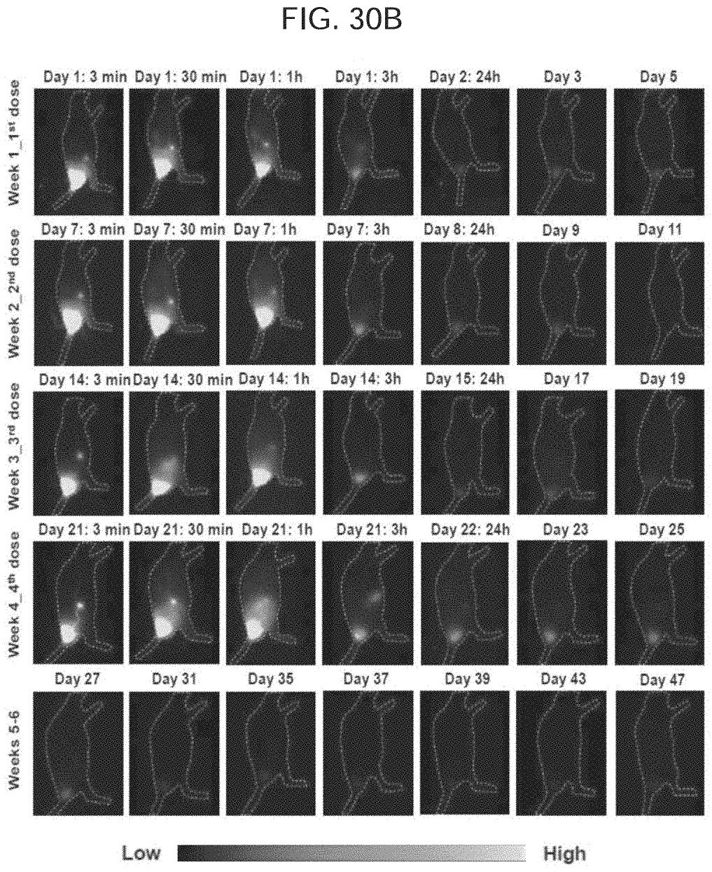

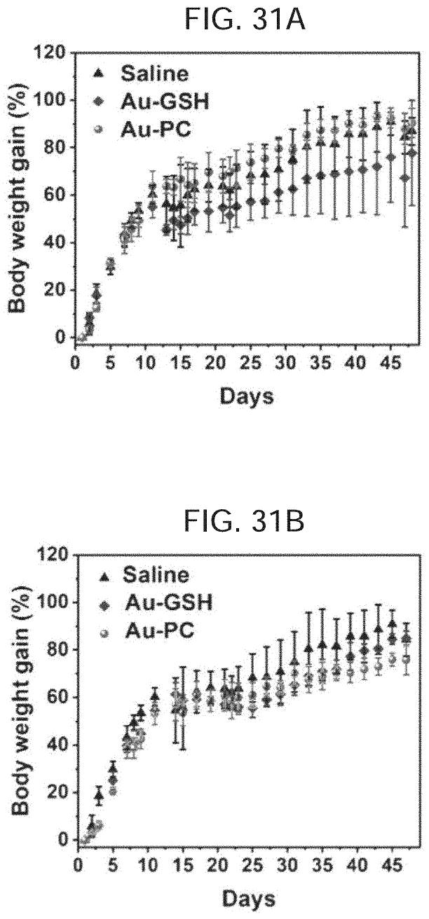

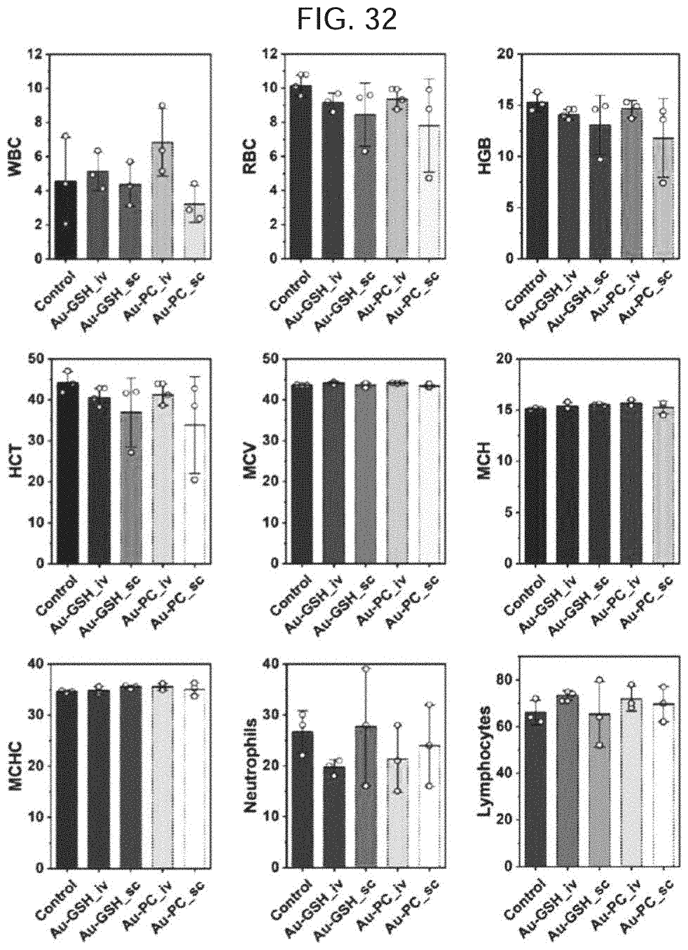

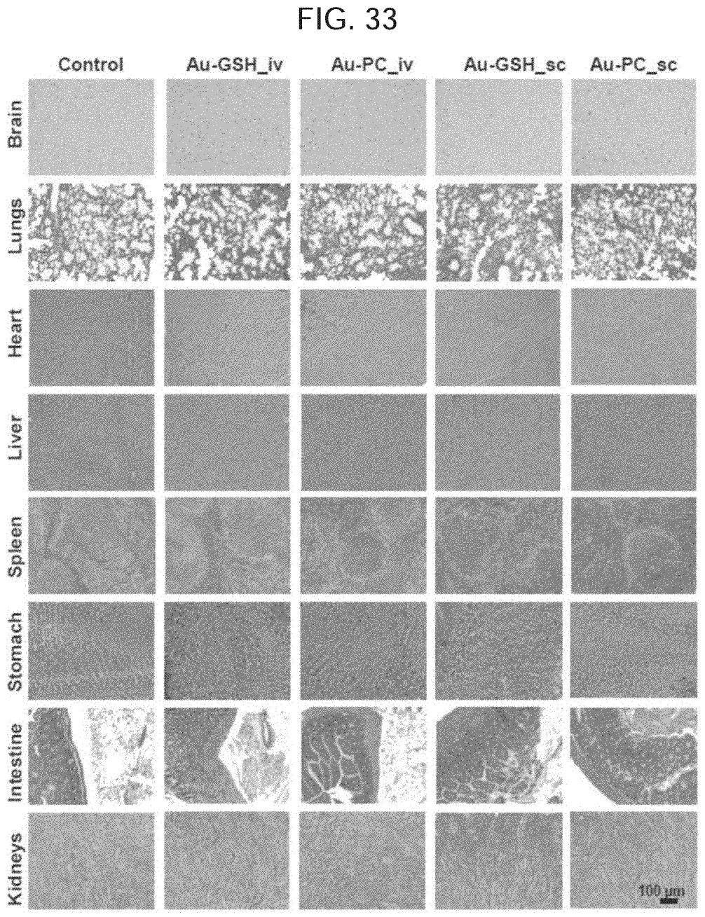

Also performed were long-term fate studies after systematic intravenous (i.v.) and subcutaneous (s.c.) administration of Au-GSH clusters (1×, ˜300 μg, FIGS. 27 and 29) and Au-PC conjugate (1×, ˜300 μg, FIGS. 28 and 30) to mice weekly (starting from three weeks old female Balb/c, n=3 in each group). The NIR-II images show fast renal clearance and LN drainage upon administration of probes. The body weight gain vs time plots show steady increase following a similar trend as the control group treated with only saline (FIG. 31). Complete blood count (CBC) analyses of blood samples collected on day 48 (i.v.) and 47 (s.c.) show no apparent toxic effects (FIG. 32). The obtained results were comparable to the control group, while small variations were due to clot present in the sample tubes. The morphology of red blood cells remained normal. Pathological examination of histological sections of hematoxylin and eosin (H&E)-stained organs show no damage at the tissue level (FIG. 33).

### Example 3—Comparing Au-PC and ICG Probes for Lymph Node Imaging in Various NIR Sub-Windows

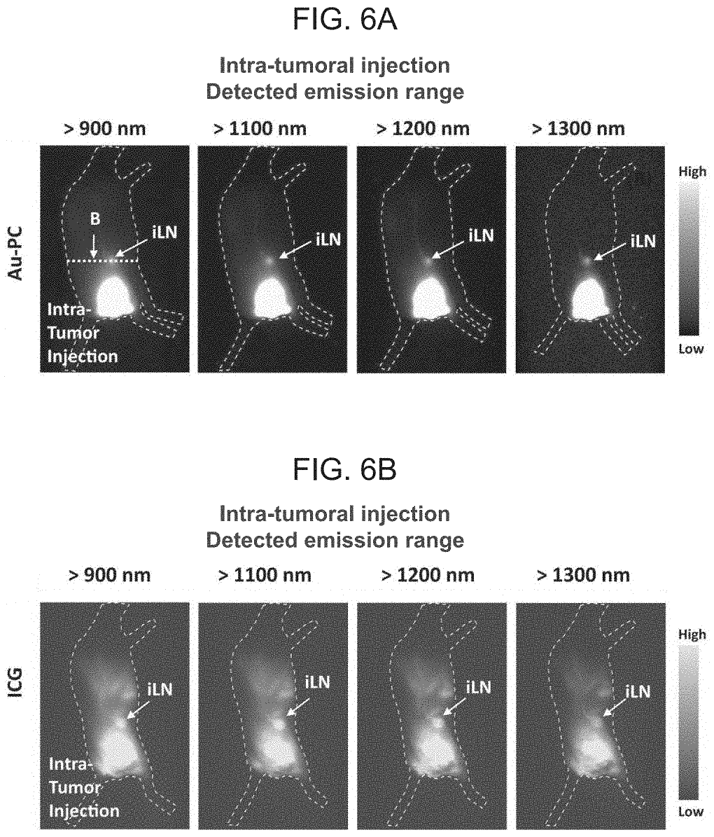

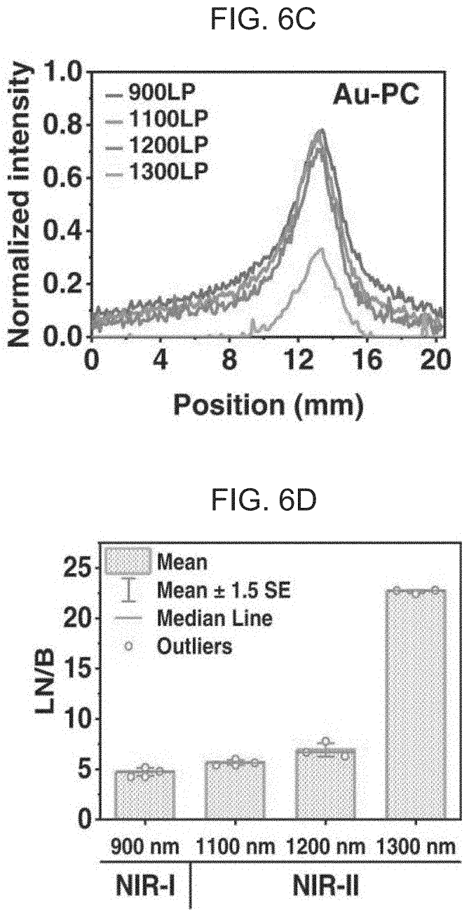

Lastly, intratumoral injection of 1× dose of Au-PC (FIG. 6A) and ICG (FIG. 6B) (5-7 weeks old female Balb/c, n=3 in each group) was performed, and LN imaging by detecting NIR-II emission of the probes (under the same 808 nm excitation) was compared at increasing wavelengths in the draining iLN at their respective peak intensity time point. The full width of half maximum (FWHM) for Au-PC and ICG-based LN imaging at >900 nm, >1100 nm, >1200 nm and >1300 nm emission windows was analyzed (FIG. 6C). With increasing the emission wavelength, the broadening of the cross-sectional profiles obviously reduced and the measured full width of half maximum (FWHM) of lymph nodes decreased from 3.8, 3.5, 3.2 to 2.8 mm (FIG. 6C), suggesting increased imaging resolution at longer emission. The LN/B ratios also increased and afforded higher LN/B ratio for Au-PC especially in the >1300 nm imaging range (FIG. 6D). The LN/B ratio measured at >1300 nm emission for Au-PC reached ˜22 (FIG. 6D), affording clear identification/imaging of the primary tier node.

### Methods

### Materials

Hydrogen tetrachloroaurate(III) trihydrate (Sigma-Aldrich, ≥99.9% trace metals basis), L-glutathione reduced (GSH, Sigma-Aldrich, ≥98.0%), sodium borohydride (Sigma-Aldrich, ≥96%), 1-(3-dimethylaminopropyl)-3-ethylcarbodiimide hydrochloride (EDC), N-hydroxysuccinimide (NHS, Thermo Scientific) and 2-Amino-2-(hydroxymethyl)-1,3-propanediol (tris-base) were used as received. DI water and indocyanine green (ICG) were purchased from Fisher Scientific. 4-Aminophenylphosphorylcholine (PC) was purchased from Santa Cruz Biotechnology Inc.

### Synthesis of Au-GSH Clusters

In a typical synthesis⁶², 5 mg of HAuCl₄·3H₂O (0.013 mmol, 1.3 mM, weigh using glass spatula) was dissolved in 10 mL DI water in a round-bottom glass flask and mixed with 16 mg of L-glutathione reduced (0.052 mmol, 5.2 mM, in 10 mL DI water) resulting in the formation of slightly milky solution. The solution was vigorously stirred for a few minutes and then upon reduction of the intermediate GSH-Au(I) complex with 5 mg freshly prepared sodium borohydride solution (0.13 mmol, 13 mM) in 10 mL water, the slightly milky-white solution immediately turned dark brown, indicating the formation of various nano-sized clusters with a common formula of Auₙ(GS)ₘ^{q}, where n is the number of gold atoms in the cluster, m is the number of glutathione ligands and q is the net charge of the cluster. The continuous etching of the reaction mixture for 24 h at room temperature resulted in the formation of the final product, i.e., Au₂₅(GS)₁₈. The purification of the sample was completed by centrifugation (4400 rpm) using 15 mL Amicon 3 K filters and water for 5-6 times. The concentrated solution was stored in 4° C. for further use (denoted as Au-GSH).

### Surface Modification and Conjugation of Au-GSH to PC Ligand

The surface modification of the cluster by PC ligands was performed using EDC/NHS chemistry. Briefly, ˜36 equivalents (of the theoretical number of —COOH groups in the cluster) of 4-aminophenylphosphorylcholine ligand were added to Au-GSH cluster (1×, 300 μg) in MES pH 7.0 buffer followed by the addition of 100 mM EDC and NHS. The conjugation was performed at room temperature on orbital shaker for 3 h and afterwards the remaining carboxylic groups from GSH were blocked by the addition of TRIS 100 mM and left to react for another hour. The final Au-GSH-PC conjugate (hereafter: Au-PC) was washed with PBS pH 7.4 buffer using Amicon 3 KDa centrifuge filters for few times and then stored in 4° C. fridge for further use.

### Characterization

UV-vis spectra were recorded on a Varian Cary 6000i UV/Vis/NIR spectrophotometer, using a quartz cuvette of 2 mm path length. Spectra were measured in the range of 200-1000 nm in water with a scanning speed of 200 nm min⁻¹ with spectral bandwidth of 2 nm. The emission spectra were measure by an Acton SP2300i spectrometer equipped with an InGaAs linear array detector (Princeton OMA-V). The quantum yields were measured using integrated sphere method. ESI-MS analyses were performed on Bruker MicroTOF-Q II; the sample was introduced by syringe pump at 3 μL/min and the full scan MS spectra were collected in negative ion mode. Inductively coupled plasma mass spectrometry (ICP-MS) was performed on a Thermo Scientific ICAP 6300 Duo View Spectrometer. The Infrared spectra were measured on Nicolet iS50 FT/IR spectrometer. The PC ligand, Au-GSH cluster and Au-PC conjugate were drop casted on a diamond internal reflection element (IRE) and allowed to dry in air. IR spectra were measured in ATR mode. The spectra were recorded with a spectral resolution of 4 cm⁻¹, in the range 400-4000 cm⁻¹.

### CryoEM Data Acquisition

3 μL Au-GSH and Au-PC samples (concentration: 6.0 μg/μL) were applied on a glow-discharged R1.2/1.3 Quantifoil grid. The grids were blotted by filter paper to remove the extra sample and quickly plunged into liquid ethane using Vitrobot Mark IV (Thermo Fisher Scientific, USA). The TEM images were collected using a Titan Krios G3 cryo-electron microscope equipped (Thermo Fisher Scientific, USA) with a K3 direct electron detector with an accelerated voltage of 300 kV. Micrographs were collected at ˜1.0 um defocus with a pixel size of 1.08 Å and electron dose of 30 e-/Å².

### Absolute Quantum Yield Measurement

The absolute quantum yields of Au-GSH and Au-PC were measured using an integrated sphere (Thorlabs; IS200). The probes were excited by an 808 nm laser and the emission was collected in the 900-1500 nm. After spreading the incoming light by an integrated sphere, the outcome light was collected using a home-built NIR spectrograph with a spectrometer (Acton SP2300i) equipped with a liquid-nitrogen-cooled InGaAs linear array detector (Princeton OMA-V). The absolute quantum yields were calculated according to the following equation:

$$Q Y = \frac{photons emitted}{photons absorbed} = \frac{E [ sample ]}{L [ blank ] - L [ sample ]}$$

where QY is the quantum yield, E[sample] is the emission intensity, and L[blank] and L[sample] are the intensities of the excitation light in the presence of the water and the NIR-II probe sample, respectively.

### Cell Viability Assay

The cytotoxicity of Au-GSH and Au-PC on 4T1 murine breast cancer (ATCC CRL-2539) and CT26 colon cancer (ATCC CRL-2638) cell lines was evaluated using MTS assay (CellTiter 96® AQueous One Solution Cell Proliferation Assay, Promega). The cell lines used in this study tested negative for mycoplasma infection. The cells were seeded at 5×10³ cells per well of 96-well plate in RPMI 1640 medium complemented with 10% FBS and 1% Penicillin-streptomycin antibiotics and left for 24 h for attachment. After incubation in a humidified atmosphere of 5% CO₂ at 37° C. for 24 h, the cells were washed twice with 200 μL base medium and afterwards varying concentrations of Au-GSH and Au-PC were added to each well, in triplicates. After 12 h of internalization, the cells were washed with medium three times and then MTS was added in each well. The absorbance was measured 4 h post incubation using Multiplate Reader (Tecan).

### Mouse Handling and Tumor Inoculation

3-9-week-old BALB/c female mice (weight: 15-20 g) were purchased from Charles River. The mice were housed on a 12-hour light/12-hour dark at ambient temperature =20-25° C. and humidity=50-65% in Stanford University's Veterinary Service Center. The bedding, nesting material, food and water were provided by the Stanford VSC facility. Prior to each experiment, the mice were shaved using hair-removing lotion (Nair, Softening Baby Oil). For in vivo imaging, the mice were anaesthetized by 2.5% isoflurane and oxygen as a carrying gas at a flow rate of 2 L/min. Per animal care protocols, the mice were carefully monitored during the imaging process and postrecovery period. The experimental groups consisted of n=3 mice. 4T1 murine breast cancer cells and CT26 colon cancer cells were inoculated on both hindlimbs of the mice and syngeneic 4T1 and CT26 tumors were grown after a few days. The animal experiments were performed when the tumor reached ˜15 mm³. The maximum allowable tumor size for a mouse bearing a single tumor or two tumors were 2.46 cm³ and 2.5 cm³.

In Vivo Wide-Field Fluorescence Imaging

The animal experiments and imaging in the NIR-II window were conducted in a two-dimensional, water-cooled 640×512 InGaAs array (Ninox 640, Raptor Photonics). The clusters were excited by an 808-nm continuous-wave diode laser at a power density of 70 mW/cm². 1100 nm long-pass filter was used in all the imaging experiments unless stated otherwise. The fluorescent probes were administered through tail vein (intravenous, i.v.), tail base (subcutaneous, s.c.) and (peri)intratumoral (i.t.). NIR-II fluorescence images were recorded at 3 min, 10 min, 30 min, 1 h, 3 h, 6 h, 24 h, 48 h p.i.

### Biodistribution of Au-GSH and Au-PC

The biodistribution of probes was analyzed 10 min, 30 min, 24 h or 48 h postadministration. The urine, feces and major organs including the liver, spleen, heart, lungs, and kidneys were collected and digested in nitric acid (68%) for 12 h. Afterwards, the solutions were heated to 150° C. in the digestion solution (nitric acid:hydrogen peroxide=4:1) until transparent and colorless solution was obtained. The concentration of gold in each sample was measured by ICP-MS (Thermo Scientific ICAP 6300 Duo View Spectrometer).

### Data Processing

LabView2009 software package was used for imaging the animals, recoding videos, and synchronously controlling laser exposure. The raw images were processed and analyzed using ImageJ 2.1. The crystallographic representation of the cluster structure was prepared using UCSF Chimera program (version 1.12) based on crystal structure data published in Reference 46.

### Statistics and Reproducibility

The graphs were prepared using Origin 2021 software package. Statistical analyses were performed using Paired comparison app available in Origin (mean comparison method: Tukey, one-sided). P values of <0.05 were considered statistically significant. Error bars represented the standard deviation (SD) of three repeated experiments. Data presented as mean values±SD. Sample sizes were chosen based on extensive experience with animal work on lymph node imaging. Each experiment was repeated at least three times. The mice were randomly selected from the cages and then divided into study groups.

### REFERENCES

1. Zahoor, S. et al. Sentinel lymph node biopsy in breast cancer: A clinical review and update. J. Breast Cancer 20, 217-227 (2017).
2. Hamdy, O., Farouk, O., El-Badrawy, A., Denewer, A. & Setit, A. Sentinel lymph node biopsy in breast cancer—an updated overview. Eur. Surg.—Acta Chirurgica Austriaca 52, 268-276 (2020).
3. Marshall, M. V et al. Near-Infrared Fluorescence Imaging in Humans with Indocyanine Green: A Review and Update. Open Surg. Oncol. J. 2, 12-25 (2010).
4. Pan, J. et al. Real-time surveillance of surgical margins via ICG-based near-infrared fluorescence imaging in patients with OSCC. World J. Surg. Oncol. 18, 96 (2020).
5. Yang, H. et al. Handheld near-infrared fluorescence imaging device using modified action cameras for peri-operative guidance of microvascular flap surgery. J. Clin. Med. 10, 1-12 (2021).
6. Yokoyama, J. et al. Long term-follow-up multicenter feasibility study of icg fluorescence-navigated sentinel node biopsy in oral cancer. Mol. Clin. Oncol. 13, 1-8 (2020).
7. van der Vorst, J. R. et al. Near-infrared fluorescence sentinel lymph node mapping of the oral cavity in head and neck cancer patients. Oral. Oncol. 49, 15-19 (2013).
8. Zhang, C. et al. Methylene Blue-Based Near-Infrared Fluorescence Imaging for Breast Cancer Visualization in Resected Human Tissues. Technol Cancer Res Treat 18, 1533033819894331 (2019).
9. Tummers, Q. R. J. G. et al. Intraoperative near-infrared fluorescence imaging of a paraganglioma using methylene blue: A case report. Int. J. Surg. Case Rep. 6, 150-153 (2015).
10. Dogan, N. U., Dogan, S., Favero, G., Köhler, C. & Dursun, P. The Basics of Sentinel Lymph Node Biopsy: Anatomical and Pathophysiological Considerations and Clinical Aspects. J. Oncol. 2019, 1-10 (2019).
11. Moncayo, V. M., Aarsvold, J. N. & Alazraki, N. P. Lymphoscintigraphy and sentinel nodes. J. Nucl. Med. 56, 901-907 (2015).
12. Vidal-Sicart, S. & Valdés Olmos, R. Using lymphoscintigraphy as a prognostic tool in patients with cancer. Res. Rep. Nucl. Med. 1, s64945 (2016).
13. Chahid, Y., Qiu, X., van de Garde, E. M. W., Verberne, H. J. & Booij, J. Risk factors for nonvisualization of the sentinel lymph node on lymphoscintigraphy in breast cancer patients. EJNMMI Res. 11, 54 (2021).
14. Jeremiasse, B. et al. Systematic review and meta-analysis concerning near-infrared imaging with fluorescent agents to identify the sentinel lymph node in oncology patients. Eur. J. Surgical Oncol. 46, 2011-2022 (2020).
15. Ballardini, B. et al. The indocyanine green method is equivalent to the 99mTc-labeled radiotracer method for identifying the sentinel node in breast cancer: A concordance and validation study. Eur. J. Surgical Oncol. 39, 1332-1336 (2013).
16. Kim, J. H., Ku, M., Yang, J. & Byeon, H. K. Recent developments of icg-guided sentinel lymph node mapping in oral cancer. Diagnostics 11, 891 (2021).
17. Welsher, K. et al. A route to brightly fluorescent carbon nanotubes for near-infrared imaging in mice. Nat. Nanotechnol. 4, 773-780 (2009).
18. Hong, G. et al. Through-skull fluorescence imaging of the brain in a new near-infrared window. Nat. Photonics 8, 723-730 (2014).
19. Wang, F. et al. In vivo NIR-II structured-illumination light-sheet microscopy. Proc. Natl. Acad. Sci. USA 118, e2023888118 (2021).
20. Bruns, 0. T. et al. Next-generation in vivo optical imaging with short-wave infrared quantum dots. Nat. Biomed. Engineer. 1,0056 (2017).
21. Wang, F. et al. In vivo non-invasive confocal fluorescence imaging beyond 1700 nm using superconducting nanowire single-photon detectors. Nat. Nanotechnol. 17, 653-660 (2022).
22. Wang, F. et al. Light-sheet microscopy in the near-infrared II window. Nat. Methods 16, 545-552 (2019).
23. Hsu, J. C. et al. Nanoparticle contrast agents for X-ray imaging applications. Wiley Interdisciplinary Rev.: Nanomed. Nanobiotechnol. 12, e1642 (2020).
24. Forte, E. et al. Radiolabeled PET/MRI nanoparticles for tumor imaging. J. Clin. Med. 9, 89 (2020).
25. Upputuri, P. K. & Pramanik, M. Photoacoustic imaging in the second near-infrared window: a review. J. Biomed. Opt. 24, 1 (2019).
26. Zhang, N.-N. et al. Recent advances in near-infrared II imaging technology for biological detection. J. Nanobiotechnol. 19, 132 (2021).
27. Yang, R. Q. et al. Surgical Navigation for Malignancies Guided by Near-Infrared-II Fluorescence Imaging. Small Methods 5, 2001066 (2021).
28. Wang, P. et al. Downshifting nanoprobes with follicle stimulating hormone peptide fabrication for highly efficient NIR II fluorescent bioimaging guided ovarian tumor surgery. Nanomed.: Nanotechnol. Biol. Med. 28, 102514 (2020).
29. Zhou, H. et al. Specific Small-Molecule NIR-II Fluorescence Imaging of Osteosarcoma and Lung Metastasis. Adv. Healthcare Mat. 9, e1901224 (2020).
30. Jacques, S. L. Optical properties of biological tissues: A review. Physics in Medicine and Biology 58, R37-R61 (2013).
31. Diao, S. et al. Biological imaging without autofluorescence in the second near-infrared region. Nano Res. 8, 3027-3034 (2015).
32. Li, Y. et al. Design of AlEgens for near-infrared IIb imaging through structural modulation at molecular and morphological levels. Nat. Commun. 11,1255 (2020).
33. Tian, R. et al. Multiplexed NIR-II Probes for Lymph Node-Invaded Cancer Detection and Imaging-Guided Surgery. Adv. Mat. 32, e1907365 (2020).
34. Andersen, C. K. et al. Carbon nanotubes—potent carriers for targeted drug delivery in rheumatoid arthritis. Pharmaceutics 13, 453 (2021).
35. Ma, Z. et al. Cross-Link-Functionalized Nanoparticles for Rapid Excretion in Nanotheranostic Applications. Angew. Chem.—Int. Ed. 59, 20552-20560 (2020).
36. Zhong, Y. et al. In vivo molecular imaging for immunotherapy using ultra-bright near-infrared-IIb rare-earth nanoparticles. Nat. Biotechnol. 37, 1322-1331 (2019).
37. Fan, Y. et al. Lifetime-engineered NIR-II nanoparticles unlock multiplexed in vivo imaging. Nat. Nanotechnol. 13, 941-946 (2018).
38. Ma, Z. et al. Near-Infrared IIb Fluorescence Imaging of Vascular Regeneration with Dynamic Tissue Perfusion Measurement and High Spatial Resolution. Adv. Fun. Mat. 28, (2018).
39. Zhang, X. D. et al. Traumatic brain injury imaging in the second near-infrared window with a molecular fluorophore. Adv. Mater. 28, 6872-6879 (2016).
40. Jiang, S. et al. Synergistic Anticancer Therapy by Ovalbumin Encapsulation-Enabled Tandem Reactive Oxygen Species Generation. Angew. Chem.—Int. Ed. 59, 20008-20016 (2020).
41. Antaris, A. L. et al. A small-molecule dye for NIR-II imaging. Nat. Mater. 15, 235-242 (2016).
42. Chakraborty, I. & Pradeep, T. Atomically Precise Clusters of Noble Metals: Emerging Link between Atoms and Nanoparticles. Chem. Rev. 117, 8208-8271 (2017).
43. Jin, R., Zeng, C., Zhou, M. & Chen, Y. Atomically Precise Colloidal Metal Nanoclusters and Nanoparticles: Fundamentals and Opportunities. Chem. Rev. 116, 10346-10413 (2016).
44. Du, B. et al. Glomerular barrier behaves as an atomically precise bandpass filter in a sub-nanometre regime. Nat. Nanotechnol. 12, 1096-1102 (2017).
45. Zheng, J., Zhang, C. & Dickson, R. M. Highly fluorescent, water-soluble, size-tunable gold quantum dots. Phys. Rev. Lett. 93, (2004).
46. Zhu, M., Eckenhoff, W. T., Pintauer, T. & Jin, R. Conversion of Anionic [Au 25 (SCH 2 CH 2 Ph) 18]—Cluster to Charge Neutral Cluster via Air Oxidation. J. Phys. Chem. C. 112, 14221-14224 (2008).
47. Aikens, C. M. Electronic and Geometric Structure, Optical Properties, and Excited State Behavior in Atomically Precise Thiolate-Stabilized Noble Metal Nanoclusters. Acc. Chem. Res. 51, 3065-3073 (2018).
48. Taylor, M. G. & Mpourmpakis, G. Thermodynamic stability of ligand-protected metal nanoclusters. Nat. Commun. 8, (2017).
49. Nonappa. Luminescent gold nanoclusters for bioimaging applications. Beilstein J. Nanotechnol 11, 533-546 (2020).
50. Xiao, Y., Wu, Z., Yao, Q. & Xie, J. Luminescent metal nanoclusters: Biosensing strategies and bioimaging applications. Aggregate 2, 114-132 (2021).
51. van de Looij, S. M. et al. Gold Nanoclusters: Imaging, Therapy, and Theranostic Roles in Biomedical Applications. Bioconjugate Chem. 33, 4-23 (2022).
52. Liu, H. et al. Atomic-Precision Gold Clusters for NIR-II Imaging. Adv. Mat. 31, (2019).
53. Li, Q., Zeman, C. J., Ma, Z., Schatz, G. C. & Gu, X. W. Bright NIR-II Photoluminescence in Rod-Shaped Icosahedral Gold Nanoclusters. Small 17, (2021).
54. Zhang, C. et al. Gold nanoclusters-based nanoprobes for simultaneous fluorescence imaging and targeted photodynamic therapy with superior penetration and retention behavior in tumors. Adv. Funct. Mater. 25, 1314-1325 (2015).
55. Li, D. et al. Gold Nanoclusters for NIR-II Fluorescence Imaging of Bones. Small 16, (2020).
56. Song, X. et al. A New Class of NIR-II Gold Nanocluster-Based Protein Biolabels for In Vivo Tumor-Targeted Imaging. Angew. Chem.—Int. Ed. 60, 1306-1312 (2021).
57. Wang, Q. et al. Biomimetic Polymer-Based Method for Selective Capture of C-Reactive Protein in Biological Fluids. ACS Appl. Mater. Interfaces 10, 41999-42008 (2018).
58. Jiang, C., Alam, M. T., Parker, S. G., Darwish, N. & Gooding, J. J. Strategies to Achieve Control over the Surface Ratio of Two Different Components on Modified Electrodes Using Aryldiazonium Salts. Langmuir 32, 2509-2517 (2016).
59. Fujiwara, N. et al. 2-Methacryloyloxyethyl phosphorylcholine (MPC)-polymer suppresses an increase of oral bacteria: a single-blind, crossover clinical trial. Clin. Oral. Investig. 23, 739-746 (2019).
60. Qi, M. et al. Graphene Oxide Thin Film with Dual Function Integrated into a Nanosandwich Device for in Vivo Monitoring of Interleukin-6. ACS Appl. Mater. Interfaces 9, 41659-41668 (2017).
61. D'Agata, R. et al. A new ultralow fouling surface for the analysis of human plasma samples with surface plasmon resonance. Talanta 221, (2021).
62. Katla, S. K., Zhang, J., Castro, E., Bernal, R. A. & Li, X. Atomically Precise Au25(SG)18 Nanoclusters: Rapid Single-Step Synthesis and Application in Photothermal Therapy. ACS Appl. Mater. Interfaces 10, 75-82 (2018).
63. Dorion, S., Ouellet, J. C. & Rivoal, J. Glutathione metabolism in plants under stress: Beyond reactive oxygen species detoxification. Metabolites 11, 641 (2021).
64. Starosolski, Z. et al. Indocyanine green fluorescence in second near-infrared (NIR-II) window. PLoS ONE 12, (2017).
65. Li, X., Fu, Y., Ma, L., Wang, Z. & Zhang, H. Spectrometric study on the interaction of indocyanine green with human serum albumin. Chem. Res. Chin. Universities 32, 343-347 (2016).
66. Chen, S., Zheng, J., Li, L. & Jiang, S. Strong resistance of phosphorylcholine self-assembled monolayers to protein adsorption: Insights into nonfouling properties of zwitterionic materials. J. Am. Chem. Soc. 127, 14473-14478 (2005).
67. Shinohara, N. et al. Long-term retention of pristine multi-walled carbon nanotubes in rat lungs after intratracheal instillation. J. Appl. Toxicol. 36, 501-509 (2016).
68. Wu, F. et al. Fluorescence imaging of the lymph node uptake of proteins in mice after subcutaneous injection: molecular weight dependence. Pharm. Res 29, 1843-1853 (2012).
69. Zeng, H. C., Hu, J. L., Bai, J. W. & Zhang, G. J. Detection of Sentinel Lymph Nodes with Near-Infrared Imaging in Malignancies. Mol. Imaging Biol. 21, 219-227 (2019).
70. Lin, J. et al. Indocyanine green fluorescence method for sentinel lymph node biopsy in breast cancer. Asian J. Surg. 43, 1149-1153 (2020).
71. Kang, X. & Zhu, M. Tailoring the photoluminescence of atomically precise nanoclusters. Chem. Soc. Rev. 48, 2422-2457 (2019).
72. Zhou, M. & Song, Y. Origins of Visible and Near-Infrared Emissions in [Au25(SR)18]-Nanoclusters. J. Phys. Chem. Lett. 12, 1514-1519 (2021).
73. Xia, N. et al. Structural Oscillation Revealed in Gold Nanoparticles. J. Am. Chem. Soc. 142, 12140-12145 (2020).
74. Pyo, K. et al. Ultrabright Luminescence from Gold Nanoclusters: Rigidifying the Au(I)-Thiolate Shell. J. Am. Chem. Soc. 137, 8244-8250 (2015).
75. Li, Q., Zeman, C. J., Schatz, G. C. & Gu, X. W. Source of Bright Near-Infrared Luminescence in Gold Nanoclusters. ACS Nano 15, 16095-16105 (2021).
76. Negishi, Y., Nobusada, K. & Tsukuda, T. Glutathione-Protected Gold Clusters Revisited: Bridging the Gap between Gold(I)-Thiolate Complexes and Thiolate-Protected Gold Nanocrystals. J. Am. Chem. Soc. 127, 5261-5270 (2005).
77. Yaghini, E. et al. In vivo biodistribution studies and ex vivo lymph node imaging using heavy metal-free quantum dots. Biomaterials 104, 182-191 (2016).
78. Choi, H. S. et al. Targeted zwitterionic near-infrared fluorophores for improved optical imaging. Nat. Biotechnol. 31, 148-153 (2013).

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

What is claimed is:

1. Gold molecular clusters functionalized with phosphorylcholine (PC) ligands.

2. The gold molecular clusters of claim 1, wherein the gold molecular clusters comprise on average from 8 to 300 gold atoms.

3. The gold molecular clusters of claim 1, wherein the gold molecular clusters are functionalized with the PC ligands via covalent linkage between the PC ligands and the gold molecular clusters.

4. The gold molecular clusters of claim 3, wherein the PC ligands are covalently linked to thiol molecules on the gold molecular clusters, wherein the thiol molecules comprise glutathione (GSH) or cysteines.

5. The gold molecular clusters of claim 1, wherein the gold molecular clusters functionalized with PC ligands are biocompatible.

6. A composition comprising the gold molecular clusters of claim 1, wherein the composition is formulated for administration to a subject.

7. A method of in vivo imaging of a tissue in a subject, the method comprising:
administering the composition of claim 6 to the subject; and
performing NIR-I (800-1000 nm) or NIR-II (1000-1700 nm) in vivo fluorescence imaging of the tissue.

8. The method according to claim 7, wherein the administering is by intravenous, subcutaneous, intra-muscular, intra-dermal, intraperitoneal intravitreal, intra-tumoral or peri-tumoral administration to the subject.

9. The method according to claim 7, wherein performing NIR-I or NIR-II in vivo fluorescence imaging of the tissue comprises detecting <1000 nm or >1000 nm fluorescence under 660 nm, 740 nm or 808 nm laser or LED excitation.

10. The method according to claim 7, wherein performing NIR-II in vivo fluorescence imaging of the tissue comprises imaging the tissue in the >1000 nm, >1100 nm, >1200 nm or >1300 nm NIR-II window.

11. The method according to claim 10, wherein performing NIR-I or NIR-II in vivo fluorescence imaging of the tissue comprises exciting the gold molecular clusters at a wavelength of from 600 nm to 850 nm.

12. The method according to claim 7, wherein the subject has cancer, and wherein the tissue is a sentinel lymph node (SLN).

13. The method according to claim 12, further comprising, subsequent to performing NIR-I or NIR-II in vivo fluorescence imaging of the SLN, performing a biopsy on the SLN to assess for cancer metastasis.

14. The method according to claim 7, wherein the tissue is a tumor.

15. The method according to claim 14, wherein the method comprises administering the composition via intra-tumor and/or peri-tumor injection, allowing the gold molecular clusters functionalized with PC ligands to infiltrate the tumor, and performing the NIR-I or NIR-II in vivo fluorescence imaging of the tumor.

16. The method according to claim 15, further comprising resecting the tumor guided by the NIR-I or NIR-II in vivo fluorescence imaging of the tumor.

17. The method according to claim 7, wherein the NIR-I or NIR-II in vivo fluorescence imaging of the tissue is performed within 3 hours of administration of the composition.

18. The method according to claim 7, wherein the gold molecular clusters functionalized with PC ligands are renally excreted from the subject within 3 days of administration of the composition.

19. A kit comprising:
the composition of claim 6; and
instructions for administering the composition to a subject for in vivo imaging of a tissue in the subject.

20. A method of synthesizing gold molecular clusters functionalized with PC ligands, the method comprising functionalizing gold molecular clusters with PC ligands.
